(19) United States
(12) Patent Application Publication
(10) Pub. No.: US 2003/0096218 A1
(43) Pub. Date: May 22, 2003

(54) Selective affinity adsorbent

(75) Inventors: Roberto Guzman, Tucson, AZ (US); Jerker Porath, Tucson, AZ (US)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: Arizona Bd of Regents/Behalf of Univ. of Arizona, Tucson, AZ

(21) Appl. No.: 10/139,207

(22) Filed: May 7, 2002

Related U.S. Application Data
(60) Provisional application No. 60/289,576, filed on May 7, 2001.

(51) Int. Cl. A01N 1/02

(52) U.S. Cl. 435/2; 435/174; 435/178; 435/179; 530/413

(57) ABSTRACT

A polymer product useful for adsorbing small molecular size solutes in the presence of large molecular size solutes comprises a matrix polymer, an affinity ligand, and a shielding ligand. The affinity ligand forms complexes with small molecular size solutes, while the shielding ligand prevents large molecular size solutes from forming complexes with the affinity ligand. The matrix polymer, affinity ligand, and shielding ligand are covalently bonded together. The affinity ligand and shielding ligand may both be independently bonded to the matrix polymer, or the affinity ligand may be bonded to the matrix polymer, and the shielding ligand bonded to the affinity ligand.

The present application claims priority to U.S. Provisional Application 60/289,576, filed May 7,2001.

### BACKGROUND OF THE INVENTION

1. Field of the Invention

The present invention relates to a polymer product for adsorption, separation and immobilization of compounds having a small molecular size, and a separation technique using the polymer product of the present invention.

2. Discussion of the Background

In biological fluids like blood, trace amounts of peptides and substances which have a small molecular size are mixed with large quantities of large-molecular size proteins. The small molecular size components often play very important roles as regulators and signaling agents in the functioning of cells. A method of efficiently extracting and separating these trace levels of small molecular size components from the bulk proteins would be extremely valuable in biochemical and environmental research, in the treatment of various medical conditions, and in the commercial scale production of peptide drugs.

It is often difficult to separate the components of complex biological mixtures using conventional chromatographic or membrane methods, because the larger sized components of these mixtures tend to clog chromatographic supports. In addition, the larger sized components of these mixtures compete with the target molecules, which often have a much smaller molecular size, for adsorptive sites on the separation medium, thereby reducing the adsorption rate and capacity of these methods. Thus, none of these conventional methods are capable of easily isolating and extracting small molecular size compounds from complex biological mixtures containing large amounts of large-molecular size components.

### SUMMARY OF THE INVENTION

The polymer product of the present invention combines, in the same separation medium, the characteristics and advantages of size exclusion and affinity adsorptive protein separation methods. The polymer product of the present invention comprises a polymeric matrix, preferably having a network structure, to which is covalently bonded an affinity ligand capable of interacting with the target small molecules, and a shielding ligand, preferably a polymer chain, covalently bonded to either the polymeric matrix or the affinity ligand, which “shields” the affinity ligand by forming a “rejection zone” around the affinity ligand, thereby preventing large molecules of a predetermined size from interacting with the affinity ligand. Thus, only molecules of an appropriate size will penetrate the “rejection” zone and interact with the affinity ligands attached to the surface of the matrix. When the polymer product of the present invention is used, for example, as a chromatographic support, the support resists clogging, and higher flow rates may be achieved, thereby increasing the speed and capacity of chromatographic separations. In addition, an improved adsorption rate and capacity for the desired biomolecules may be obtained.

When the polymer product of the present invention is used as a chromatographic support it has both adsorptive and size exclusion properties, and therefore separates mixtures by a combination of adsorption and permeation chromatography.

### BRIEF DESCRIPTION OF DRAWINGS

FIG. 1 is a schematic representation of the polymer product of the present invention.

FIG. 2 is a schematic representation of two embodiments of the polymer product of the present invention.

FIG. 3 is a schematic representation of an extracorporeal blood perfusion device employing the polymer product of the present invention.

FIG. 4 is a plot of the copper capacity of various NOVAROSE-IDA/PEG—CH₃ adsorbents as a function of the amount of PEG—CH₃ bonded to the polymeric matrix of the adsorbent.

FIG. 5 is a plot of the frontal analysis of NOVAROSE-IDA (adsorbent #1) with a solution of cytochrome-c (1 mg/ml; A₂₈₀=1.677) at a column volume of 0.55 ml and a flow rate of 1 cm/min.

FIG. 6 is a plot of the frontal analysis of NOVAROSE-IDA/PEG—CH₃ 20 μmol/g (adsorbent #2) with a solution of cytochrome-c (1 mg/ml; A₂₈₀=1.627) at a column volume of 0.59 ml and a flow rate of 1 cm/min.

FIG. 7 is a plot of the frontal analysis of NOVAROSE-IDA/PEG—CH₃ 50 μmol/g (adsorbent #3) with a solution of cytochrome-c (1 mg/ml; A₂₈₀=1.534) at a column volume of 0.59 ml and a flow rate of 1 cm/min.

FIG. 8 is a plot of the frontal analysis of NOVAROSE-IDA/PEG—CH₃ 100 μmol/g (adsorbent #4) with a solution of cytochrome-c (1 mg/ml; A₂₈₀=1.534) at a column volume of 0.57 ml and a flow rate of 1 cm/min.

FIG. 9 is a plot of the frontal analysis of NOVAROSE-IDA/PEG—CH₃ 200 μmol/g (adsorbent #5) with a solution of cytochrome-c (1 mg/ml; A₂₈₀=1.534) at a column volume of 0.59 ml and a flow rate of 1 cm/min.

FIG. 10 is a plot of the frontal analysis of NOVAROSE-IDA (adsorbent #1) with a solution of RNase A (1 mg/ml; A₂₈₀=0.492) at a column volume of 0.57 ml and a flow rate 1 of cm/min.

FIG. 11 is a plot of the frontal analysis of NOVAROSE-IDA/PEG—CH₃ 20 μmol/g (adsorbent #2) with a solution of RNase A (1 mg/ml; A₂₈₀=0.492) at a column volume of 0.55 ml and a flow rate of 1 cm/min.

FIG. 12 is a plot of the frontal analysis of NOVAROSE-IDA/PEG—CH₃ 50 μmol/g (adsorbent #3) with a solution of RNase A (1 mg/ml; A₂₈₀=0.508) at a column volume of 0.96 ml and a flow rate of 1 cm/min.

FIG. 13 is a plot of the frontal analysis of NOVAROSE-IDA/PEG—CH₃ 100 μmol/g (adsorbent #4) with a solution of RNase A (1 mg/ml; A₂₈₀=0.541) at a column volume of 0.57 ml and a flow rate of 1 cm/min.

FIG. 14 is a plot of the frontal analysis of NOVAROSE-IDA/PEG—CH₃ 200 μmol/g (adsorbent #6) with a solution of RNase A (1 mg/ml; A₂₈₀=0.534) at a column volume of 0.59 ml and a flow rate of 1 cm/min.

FIG. 15 is a plot of the frontal analysis of NOVAROSE-IDA (adsorbent #1) with a solution of albumin (1 mg/ml; A₂₈₀=0.632) at a column volume of 0.59 ml and a flow rate of 1 cm/min.

FIG. 16 is a plot of the frontal analysis of NOVAROSE-IDA/PEG—CH₃ 20 μmol/g (adsorbent #2) with a solution of albumin (1 mg/ml; A₂₈₀=0.554) at a column volume of 0.57 ml and a flow rate of 1 cm/min.

FIG. 17 is a plot of the frontal analysis of NOVAROSE-IDA/PEG—CH₃ 50 μmol/g (adsorbent #3) with a solution of albumin (1 mg/ml; A₂₈₀=0.646) at a column volume of 0.97 ml and a flow rate of 1 cm/min.

FIG. 18 is a plot of the frontal analysis of NOVAROSE-IDA/PEG—CH₃ 100 μmol/g (adsorbent #4) with a solution of albumin (1 mg/ml; A₂₈₀=0.642) at a column volume of 0.57 ml and a flow rate of 1 cm/min.

FIG. 19 is a plot of the frontal analysis of NOVAROSE-IDA/PEG—CH₃ 200 μmol/g (adsorbent #6) with a solution of albumin (1 mg/ml; A₂₈₀=0.646) at a column volume of 0.59 ml and a flow rate of 1 cm/min.

FIG. 20 is a reverse phase chromatogram of LDH isolated from chicken breast muscle after ultrafiltration.

FIG. 21 is a reverse phase chromatogram of fragments obtained after cyanogen bromide cleavage of LDH.

FIG. 22 is a reverse phase chromatogram of LDH fragments in 60 mM imidazole after centrifugation and filtration.

FIG. 23 is a reverse phase chromatogram of the breakthrough peak from LDH peptides on Chelating SEPHAROSE FF.

FIG. 24 is a reverse phase chromatogram of the elution peak from LDH peptides on Chelating SEPHAROSE FF.

FIG. 25 is a reverse phase chromatogram of the breakthrough peak from LDH peptides on NOVAROSE-IDA (adsorbent #1).

FIG. 26 is a reverse phase chromatogram of the elution peak of a solution of LDH peptides on NOVAROSE-IDA (adsorbent #1).

FIG. 27 is a reverse phase chromatogram of the breakthrough of a solution of LDH peptides on NOVAROSE-IDA/PEG—CH₃ 20 μmol/g (adsorbent #2).

FIG. 28 is a reverse phase chromatogram of the first elution tube of a solution of LDH peptides on NOVAROSE-IDA/PEG—CH₃ 20 μmol/g (adsorbent #2).

FIG. 29 is a reverse phase chromatogram of the second elution tube of a solution of LDH peptides on NOVAROSE-IDA/PEG—CH₃ 20 μmol/g (adsorbent #2).

FIG. 30 is a reverse phase chromatogram of the breakthrough peak of a solution of LDH peptides on NOVAROSE-IDA/PEG—CH₃ 50 μmol/g (adsorbent #3).

FIG. 31 is a reverse phase chromatogram of the first elution tube of a solution of LDH peptides on NOVAROSE-IDA/PEG—CH₃ 50 μmol/g (adsorbent #3).

FIG. 32 is a reverse phase chromatogram of the second elution tube of a solution of LDH peptides on NOVAROSE-IDA/PEG—CH₃ 50 μmol/g (adsorbent #3).

FIG. 33 is a reverse phase chromatogram of the breakthrough peak of a solution of LDH peptides on NOVAROSE-IDA/PEG—CH₃ 100 μmol/g (adsorbent #4).

FIG. 34 is a reverse phase chromatogram of the elution of a solution of LDH peptides on NOVAROSE-IDA/PEG—CH₃ 100 μmol/g (adsorbent #4).

FIG. 35 is a reverse phase chromatogram of the breakthrough peak of a solution of LDH peptides on NOVAROSE-IDA/PEG—CH₃ 50 μmol/g (adsorbent #3) at pH 7.5.

FIG. 36 is a reverse phase chromatogram of the elution of a solution of LDH peptides on NOVAROSE-IDA/PEG—CH₃ 50 μmol/g (adsorbent #3) at pH 7.5.

FIG. 37 is a reverse phase chromatogram of the breakthrough peak of a solution of LDH peptides on NOVAROSE-IDA/PEG—CH₃ 50 μmol/g (adsorbent #3) in the absence of imidazole.

FIG. 38 is a reverse phase chromatogram of the elution of a solution of LDH peptides on NOVAROSE-IDA/PEG—CH₃ 50 μmol/g in the absence of imidazole.

FIG. 39 is a reverse phase chromatogram of the breakthrough of a solution of LDH peptides on NOVAROSE-IDA/PEG—CH₃ 50 μmol/g (adsorbent #3) at a flow rate of 0.33 cm/min.

FIG. 40 is a reverse phase chromatogram of the first elution tube of a solution of LDH peptides on NOVAROSE-IDA/PEG—CH₃ 50 μmol/g (adsorbent #3) at a flow rate of 0.33 cm/min.

FIG. 41 is a reverse phase chromatogram of the second elution tube of a solution of LDH peptides on NOVAROSE-IDA/PEG—CH₃ 50 μmol/g (adsorbent #3) at a flow rate of 0.33 cm/min.

FIG. 42 is a reverse phase chromatogram of the breakthrough of a solution of LDH peptides on NOVAROSE-IDA/PEG—CH₃ 50 μmol/g (adsorbent #3) at a flow rate of 2 cm/min.

FIG. 43 is a reverse phase chromatogram of the first elution tube of a solution of LDH peptides on NOVAROSE-IDA/PEG—CH₃ 50 μmol/g (adsorbent #3) at a flow rate of 2 cm/min.

FIG. 44 is a reverse phase chromatogram of the second elution tube of a solution of LDH peptides on NOVAROSE-IDA/PEG—CH₃ 50 μmol/g (adsorbent #3) at a flow rate of 2 cm/min.

FIG. 45 is a reverse phase chromatogram of the breakthrough of a solution of LDH peptides on NOVAROSE-IDA/PEG—CH₃ 50 μmol/g (adsorbent #3) at a flow rate of 2 cm/min.

FIG. 46 is a reverse phase chromatogram of the first elution tube of a solution of LDH peptides on NOVAROSE-IDA/PEG—CH₃ 50 μmol/g (adsorbent #3) with 0.25 M NaCl.

FIG. 47 is a reverse phase chromatogram of the second elution tube of a solution of LDH peptides on NOVAROSE-IDA/PEG—CH₃ 50 μmol/g (adsorbent #3) with 0.25 M NaCl.

FIG. 48 is a size exclusion chromatogram of human plasma diluted tem-fold in 20 mM NaPO₄ with 0.25 M NaCl at a pH of 7.45.

FIG. 49 a size exclusion chromatogram of human plasma diluted ten-fold in 20 mM NaPO₄ with 0.25 M NaCl containing 15 micromoles of copper.

FIG. 50 is a size exclusion chromatogram of the breakthrough of human plasma diluted ten-fold in a solution containing copper on NOVAROSE-IDA/PEG—CH₃ 200 μmol/g (adsorbent #6).

FIG. 51 is a size exclusion chromatogram of the elution of human plasma diluted ten-fold with a solution containing copper on NOVAROSE-IDA/PEG—CH₃ 50 μmol/g (adsorbent #3).

FIG. 52 is a size exclusion chromatogram of an elution buffer containing 0.2% copper.

FIG. 53 is a size exclusion chromatogram of an elution buffer.

FIG. 54A is a size exclusion chromatogram of the eluted material from a control gel (adsorbent #7).

FIG. 54B is a size exclusion chromatogram of the eluted material from adsorbent #8.

FIG. 54C is a size exclusion chromatogram of the eluted material from adsorbent #9.

FIG. 54D is a size exclusion chromatogram of the eluted material from adsorbent #10.

### DETAILED DESCRIPTION OF THE INVENTION

The polymer product of the present invention comprises a polymeric matrix, an affinity ligand, and a shielding ligand. The novel polymeric matrix of the present invention is a solid or water soluble polyhydroxylated polymer that preferably forms a matrix having a network structure. The polymeric matrix is substituted with molecular affinity ligands and shielding ligands preferably comprising polyalkylene ether chains terminated with neutral groups to control the permeation of molecules into the surface of the polymeric matrix. By forming molecular-scale steric barriers, the polyalkylene ether chains “shield” the affinity ligand by providing a molecular-scale obstacle which hinders large molecular size solutes in a solution from diffusing into and permeating the polymeric matrix, and thereby blocking access to the molecular affinity ligands. In this way, the polymeric product of the present invention preferentially adsorbs small molecular size solutes.

The polymer product of the present invention has a structure that can be schematically depicted as:

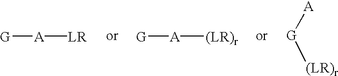

where “G” is the polymeric matrix forming a support for the molecular affinity ligand “A” and the shielding ligand “L”. The polymeric matrix “G” may be either a solid or a soluble polymer phase such as a gel or membrane. The gel or membrane may comprise an insoluble polysaccharide such as, for example, cellulose, cross-linked dextran, cross-linked agar or agarose. If the polymeric matrix is a cross-linked agar or agarose, the agar or agarose may be chemically functionalized, for example with an oxirane or halohydrin. Such materials are sometimes termed “activated” agars. The polymeric matrix of the present invention may also comprise a cross-linked polyamine such as polyethyleneimine or a hybrid comprising a polyamine chemically linked to an insoluble polysaccharide. The polymeric matrix of the present invention may also comprise a derivative or degradation product of a hybrid gel. The polymeric matrix may also be a liposome, in which the matrix consists of a membrane formed from phospholipids.

The molecular affinity ligand “A” may include any conventional molecular affinity ligands known to interact with small molecules of interest, such as metal ions, metal ion complexes, small proteins, peptides, immunoglobulins, microglobulins, antibodies, and their degradation products, nucleic acids, antibiotics and other secondary metabolites, toxins, drugs, hormones, and biotoxins. The molecular affinity ligands of the present invention may include, for example, chelating agents such as IDA (iminodiacetic acid) and polyamine derived chelators such as TREN (tris(2-aminoethyl)amine), hydroxy aromatics such as salicylidene derivatives derived from salicyl aldehydes, sulfide and sulfone groups, and various combinations of such groups. The molecular affinity groups of the present invention may also include metal complexes with any of the above groups.

The molecular affinity ligands may be attached by any conventional manner to the polymeric matrix of the present invention, preferably by covalent bonding. For example, the affinity ligand may be bonded directly to the polymeric matrix, or may be attached to a shielding ligand (i.e., “L” as discussed below) bonded to the polymeric matrix.

The shielding ligand “L” may be bonded to the affinity ligand or directly to the polymeric matrix. The shielding ligand is preferably a polyalkylene ether chain. The polyalkylene ether chain may be a homopolymer or copolymer, and may include, for example, ethylene oxide, propylene oxide, butylene oxide, or tetramethylene oxide repeating units, or various combinations of these repeat units. If the polyalkylene ether chain is a copolymer, it may be a block copolymer, a random copolymer, or a graft copolymer. In addition, the polyalkylene ether chain may include functional groups, such as amine, amide, ester, sulfide, sulfoxide, sulfone, sulfinate or sulfonate groups, which may be derived from functional groups used to graft the polyalkylene ether chain to the affinity ligand or network matrix.

“R” is a neutral ligand attached to the terminal end of the shielding ligand “L” and may include groups conventionally used to form terminal groups on, for example, polyalkylene ether chains. For example, the neutral ligand “R” may be a group such as methyl, ethyl, propyl, phenyl, substituted phenyl, and trialkylsilyl (e.g., trimethylsilyl, triethylsilyl, triphenylsilyl, etc.). By “neutral”, we mean that the group “R” is not an affinity ligand itself, and does not interact with solutes in the solutions that come in contact with the polymer product of the present invention. The symbol “r” represents the number of “L” shielding ligands attached to the affinity ligand or polymeric matrix. For example, r may have an integer value of 1, 2, etc.

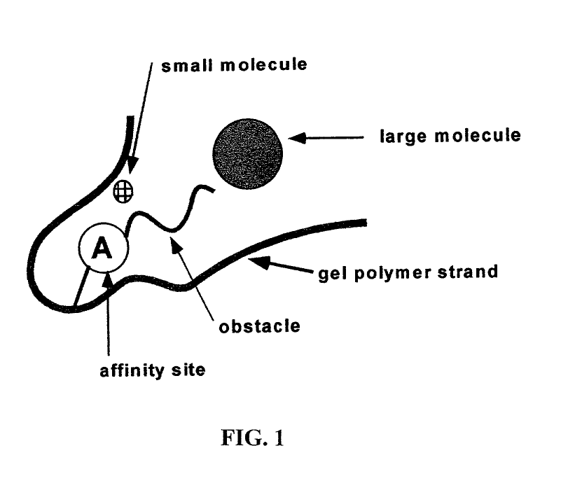

In use, the polymer product of the present invention may be represented as shown in FIG. 1. The polymeric matrix (i.e., “G”) may be comprised of numerous entangled or crosslinked polymer strands that define a porous polymeric gel matrix. A molecular affinity ligand “A” covalently attached to the polymer strand may be disposed, for example, in a pore of the polymeric gel matrix, into which a mixture of large and small molecules (depicted in FIG. 1, respectively, as large and small circles) diffuse. The large molecules are not able to effectively interact with the molecular affinity ligand due to the steric barrier provided by the shielding ligand attached to the molecular affinity ligand, whereas small molecules may selectively diffuse into proximity with the molecular affinity ligand. Thus, the polymer product of the present invention is able to selectively adsorb small molecules in the presence of large molecules.

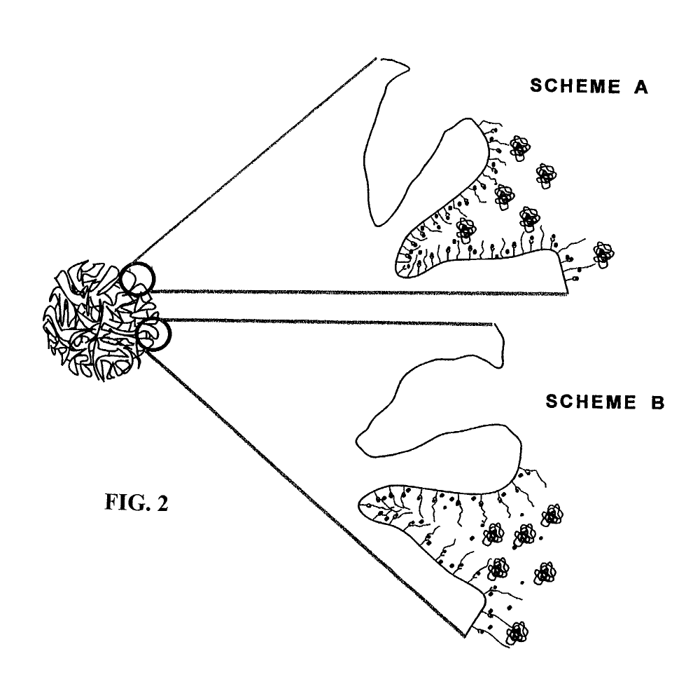

In FIG. 2, the various ways in which the affinity ligand “A” and polyalkylene ether chain “L” may be combined on the matrix support are described schematically. In Scheme A, the affinity ligands and shielding ligands are each separately attached (i.e., covalently bonded) directly to the polymeric matrix. In Scheme B, the affinity ligand is attached to the shielding ligand, which is in turn attached to the polymeric matrix. In both schemes, the larger molecules cannot easily penetrate into the pores of the polymeric matrix due to the steric barrier formed by the shielding ligand, and thus do not interfere or compete for binding sites with smaller size solutes. Smaller size solutes are thus selectively adsorbed.

Particularly preferred polymer products according to the present invention, have molecular affinity ligands which are located close to the polymeric matrix and are totally or partially blocked by the steric hindrance provided by the shielding ligand, and are particularly suitable for selectively adsorbing peptides and small molecular size proteins. This structure prevents large-size protein molecules from coming into contact with the molecular affinity ligands, so that only peptides and other molecules below a certain size can reach the adsorption site to form an adsorption complex. After the desired molecules are attached to the solid polymeric phase (i.e., the polymer product of the present invention) by means of these affinity complexes, the large molecular size components of the mixture, independently of their affinity to the affinity ligand, as well as small components which do not have affinity for the affinity ligand, are not retained and may be washed away. The adsorbed components of the appropriate size and affinity may then be desorbed from the polymeric matrix, and separated from the original mixture.

The molecular affinity ligand and/or shielding ligand (e.g., polyalkylene ether chains) may be prepared by various methods, as described below:

### EXAMPLES

Various embodiments of the polymer product of the present invention, and a method of making and using the polymer product of the present invention are described in detail below. Obviously, numerous modifications and variations on the present invention are possible in light of the teachings of the present specification. It is therefore to be understood that within the scope of the appended claims, the invention may be practiced otherwise than as specifically described herein.

Examples of Synthesis According to the Present Invention

1) Polyethyleneglycol (PEG) monomethyl ether may be heated with thionyl bromide (or thionyl chloride) to form a brominated (or chlorinated) PEG monomethyl ether:

CH₃(OCH₂—CH₂)ₙ—OH+SOBr₂→CH₃(OC H₂—CH₂—)ₙ—Br

The bromo derivative may be treated with triethylenetetramine (TREN):

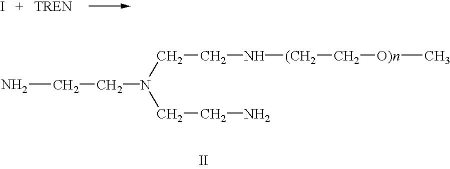

with excess TREN II may also be obtained:

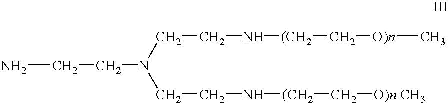

II (or/and III) may then be coupled to activate agar, signified by the formula

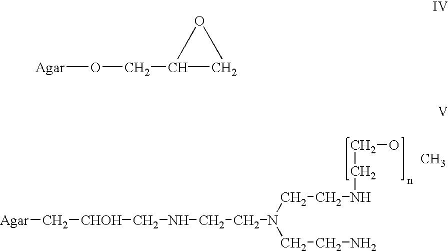

V is an example of one embodiment of the polymer product of the present invention. The TREN residue may form a metal chelate, for example with Cu²⁺ or Pd²⁺. The metal chelate may also form a strong adsorption site for peptides and proteins and provides stronger affinity for the metal ions. The PEG-residue acts as the shielding ligand that prevents large-size solutes from approaching the chelate.

### Example 2

Oxirane or halohydrin activated agar may be converted to a thiol gel. For example:

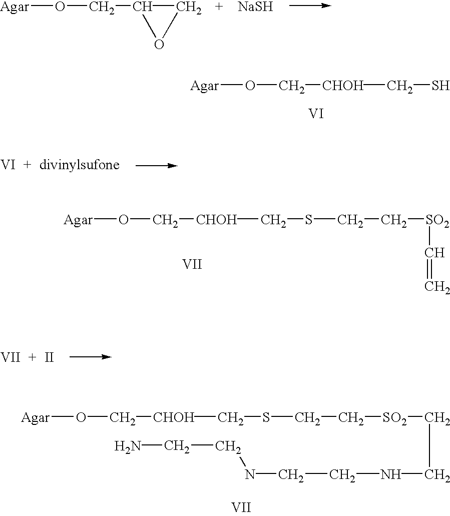

As described above for the modified agar V, the modified agar VII may also be chelated with metal ions (e.g., Cu²⁺ and Pd²⁺), and in its chelated form can act as an affinity ligand.

In compound VIII, the affinity ligand (i.e., adsorption site), as in Example 1, consists of a metal chelating group, but compound VIII also contains a group having the structure —S—CH₂—CH₂—SO₂—. In the presence of high concentration of antichaotropic salts such as K₂SO₄ this group shows affinity for certain specific proteins (thiophilic adsorption), and is therefore useful as an affinity ligand (i.e., adsorbent) for immunoglobulins and their degradation products of small molecular size.

### Example 3

V may be further derivatized to increase the activity of the affinity ligand (i.e., adsorption site). For example, V may be condensed with an aromatic aldehyde such as salicyl aldehyde to form

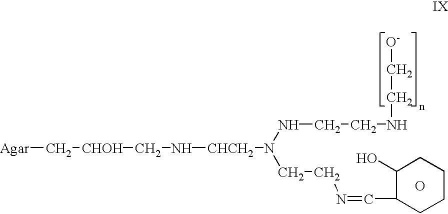

The salicylidene (“salene”) derivative IX is a strong metal chelating group. After chelating to a metal, the chelated salene derivative IX strongly binds proteins having an affinity for the metal ion bound to IX.

As discussed above, the efficiency by which the polymer product of the present invention excludes large-molecular size molecules from binding to affinity ligand depends on steric factors:

1) The molecular size of the shielding ligand forming a molecular “obstacle” to exclude large molecular size molecules from binding to the affinity ligand. For example, the steric properties of the polyalkylene ether chain may depend on the number of alkylene ether repeat units, n, in, e.g., the PEG-residue —(CH₂—CH₂—O—)n. In addition, if there are two PEGs per adsorption sites as in compound III, the steric hindrance provided by the two PEG chains is greater than the steric hindrance provided by a single PEG chain.

2) The density of the network formed by the polymeric matrix. A dense polymeric matrix provides smaller “openings” between the polymer chains of the matrix polymer, thereby excluding molecules having a molecular size which is greater than such “openings.”

3) The molecular size of the solutes. Solutes which are larger that the effective size of the openings in the network of the polymeric matrix, or which are large relative to the size of the shielding ligand (e.g., polyalkylene ether chain), are more effectively blocked from interaction with the affinity ligand than are solutes having a smaller molecular size.

By taking each of the above factors into account, the polymer product of the present invention may be modified to optimize its performance for a particular molecular size range of solutes.

The polymer product of the present invention is suitable for different application in which it is necessary to separate small molecular size solute molecules from mixtures containing large molecular size solutes. For example, the polymer product of the present invention may be used to remove metal ions and small metal ion complexes from aqueous solutions. It may be used for removing undesirable substances in blood such as small molecular size immunoglobulins, microglobulins, antibodies and their degradation products. Low molecular size antibiotics and other secondary metabolites may be size-selectively adsorbed and recovered from bacterial cultures.

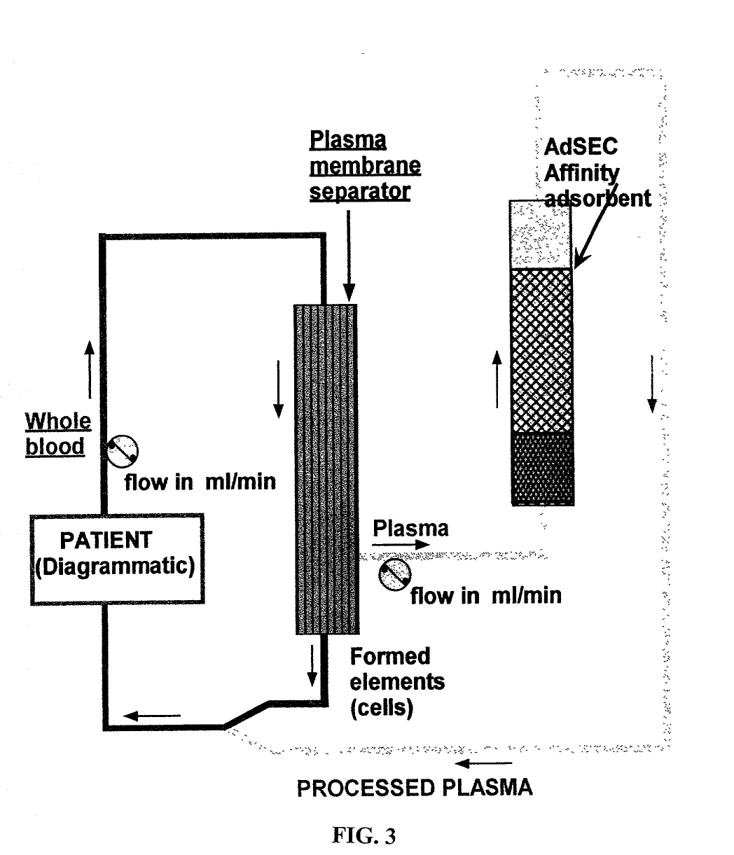

Another feasible and practical use of the polymer product of the present invention is in extracorporeal perfusion systems for blood. For example, the polymer product of the present invention may be used to remove copper and peptides from blood. A schematic of an example of such a system is shown in FIG. 3. The polymer product of the present invention may be used in any perfusion or blood dialysis system by contacting the blood or a component of the blood with a gel or membrane comprising the polymer product of the present invention.

Synthesis and Properties of NOVAROSE IDA/PEG—CH₃ Adsorbents for Polymer Modulated Controlled Permeation

General Procedure

Aminomonomethoxy-PEG was coupled to commercial NOVAROSE gel in an end-on configuration. By “end-on configuration” we mean that the terminal amino group of the aminomonomethoxy-PEG reacts with the activated surface of the NOVAROSE gel, thereby covalently bonding the PEG to a polymer chain of the NOVAROSE gel so that the PEG chain extends essentially normal to the surface of the gel. (Depending on the functionality of the shielding ligand, the shielding ligand may also be attached to the gel in a “side-on configuration.) In this scheme the free methoxy group will not interact with proteins or bound metal in a detrimental manner. Aminomonomethoxy-PEG (5000 D) has been widely studied for protein rejection capability on flat surfaces, for example in Osterberg, E., et al. (1995), “Protein-Rejection Ability of Surface-Bound Dextran in End-On and Side-On Configurations:Comparison to PEG,” J. Biomed. Mater.Res.29:741-747; Osterberg, E., et al. (1993), “Comparison of Polysaccharides and Poly(ethylene glycol) Coatings for Reduction of Protein Adsorption on Polystyrene Surfaces.” Colloids Surfaces A: Physicochem. Eng. Aspects 77: 159-169; Holmberg, K. et al. (1993) “Effects on Protein Adsorption, Bacterial Adhesion and Contact Angle of Grafting PEG Chains to Polystyrene,” J. Adhesion Sci. Technol. 7: 503-517, each of which is herein incorporated by reference. Osterberg et al. (1995) determined that a dense coating of 195-350 Å per PEG molecule resulted in a flat surface coating with a thickness on the magnitude of 100 Å.

Different variants of the polymer product of the present invention were characterized using frontal analysis of cytochrome-c, ribonuclease A, and albumin on an immobilized copper column. (Frontal analysis is a mode of operation in which a solute of mixture of solutes is continuously fed into a column until a concentration profile is developed.) Cu(II) and Ni (II) were used to study the retention capabilities of a model peptide mixture containing a 3400 Dalton nickel binding peptide. Selected variants of the polymer product were selected for optimization of peptide binding and the ability to extract only metal from protein mixtures.

Materials

NOVAROSE 100/40 ACT^{high} (i.e., cross-linked agarose) was obtained from Inovata AB (Bromma, Sweden). NH₂—PEG—CH₃ (aminomonomethoxy-PEG) with an average molecular weight of 5000 Daltons was synthesized according to the method described in Birkenmeier, G et al. (1991), “Immobilized Metal Ion Affinity Partitioning, a Method Combining Metal-Protein Interaction and Partitioning of Proteins in Aqueous Two-Phase Systems,” J. Chromatography 539: 267-277, herein incorporated by reference. Iminodiacetic acid (IDA), glycine, ethylenediaminetetraacetic acid (EDTA) trisodium salt, and imidazole (1,3-diaza-2,4-cycclopentadiene) were acquired from Sigma (St. Louis, Mo.).

Cytochrome-c- from bovine heart; ribonuclease A (RNase A), Type I-AS from bovine pancreas; and albumin, bovine, fraction V, were also obtained from Sigma (St. Louis, Mo.). Human plasma was acquired from the American Red Cross (Tucson, Ariz.).

Frozen chicken breast was purchased from a supermarket. Trifluroacetic acid and cyanogen bromide were obtained from Aldrich (Milwaukee, Wis.). SEPHADEX G-25 (medium) and chelating SEPHAROSE FF were acquired from Pharmacia Biotech (Piscataway, N.J.). Acetonitrile was purchased from Burdick & Jackson (Muskegon, Mich.). A PEP RPC 218TP column was acquired from Vydac (Hesperia, Calif.) and a BIO-SILECT SEC 400-5 column was purchased from Bio-Rad (Hercules, Calif.). All other chemicals utilized were of analytical or reagent grade.

Synthesis of NOVAROSE-IDA/PEG—CH₃ Adsorbents.

Six variants of adsorbents (i.e., polymer products) according to the present invention were prepared, each having different ratios of IDA and aminomonomethoxy-PEG. 12 g. of suction-dried NOVAROSE 100140 ACT^{high} was divided equally into six 50 ml conical tubes and 4 ml of 1.0 M Na₂CO₃ were added to each tube. To tube #1, 20 ml of 3% IDA in 1.0 M Na₂CO₃ with a pH adjusted to over 12 using 10 M NaOH was added. The adsorbent prepared in this manner served as the control for this experiment, and did not have a shielding ligand.

20 ml of 1% NH₂—PEG—CH₃ (20 μmol/g gel) in 1.0 M Na₂CO₃ at a pH≧12 were added to tube #2. 20 ml of 2.5 % NH₂—PEG—CH₃ (50 μmol/g gel) in 1.0 M Na₂CO₃ at a pH≧12 were added to tube #3. 20 ml of 5.0% NH₂—PEG—CH₃ (100 μmol/g gel) in 1.0 M Na₂CO₃ at a pH≧12 were added to tube #4. 20 ml of 7.5% NH₂—PEG—CH₃ (150 μmol/g gel) in 1.0 M Na₂CO₃ at a pH≧12 were added to tube #5. 20 ml of 10% NH₂—PEG—CH₃ (200 μmol/g gel) in 1.0 M Na₂CO₃ at a pH≧12 were added to tube #6. The final pH of the coupling reaction in each tube was adjusted to pH≧12 using 1.0 M NaOH, and the tubes were shaken at room temperature for 24 hours.

After this first reaction step, every tube other than the control (# 1) was subsequently removed from the shaker, repeatedly washed with 1.0 M Na₂CO₃, resuspended in 4 ml 1.0 M Na₂CO₃ and transferred to a 50 ml conical tube. To each of these five tubes, 20 ml of 3% IDA in 1.0 M Na₂CO₃ at a pH≧12 were added. The final pH of each mixture was adjusted to a pH≧12 and the tubes were shaken at room temperature for 24 hours.

After this second reaction step, each tube was removed from the shaker, thoroughly washed with 1.0 M Na₂CO₃, re-suspended in 4 ml 1.0 M Na₂CO₃, and transferred to a 50 ml conical tube. To each of these six tubes, 20 ml of 2.5% glycine in 1.0 M Na₂CO₃ at a pH≧12 was added. The final pH of each mixture was adjusted to a pH≧12 and the tubes were shaken at room temperature for 24 hours.

Finally, each tube was removed from the shaker and sequentially washed with deionized water, 1.0 M NaOH, deionized water, 0.1 M HCl, and deionized water until a neutral pH was obtained. Each adsorbent thus prepared was stored in 20% ethanol in the form of a gel until utilized.

Measurement of Copper Capacity.

Each adsorbent according to the present invention, prepared as described above was packed in a 3.4×0.5 cm I.D. column. The columns were packed as follows. A slurry of the adsorbent was prepared in deionized water. Small aliquots of the slurry were slowly poured into the column, and allowed to stand to minimize or prevent the formation of air bubbles in the column. After packing, each column was thoroughly washed with 10 column volumes of deionized water at a flow rate of 1 cm/min (0.2 ml/min). A selected volume of either 50 mM or 20 mM copper sulfate solution was loaded onto each column, based on the anticipated copper capacity of the adsorbent. Each column was again washed with 10 column volumes of deionized water, resulting in a distinct immobile blue phase on the adsorbent of each column. The copper capacity of each adsorbent was calculated based on the volume of adsorbent that was colored blue by the adsorbed copper, and the known concentration of copper solution loaded on the column. For example, since the column diameter was 0.5cm, a 1 cm length of blue-colored adsorbent had a volume of 0.2 ml.

Protein Capacity Determination

The protein capacity of each adsorbent according to the present invention was analyzed by frontal analysis according to methods previously described in Belew, M. et al. (1987)s, “Interaction of Proteins with Imobilized Cu(II): Quantitation of Adsorption Capacity, Adsorption Isotherms and Equilibrium Constants by Frontal Analysis,” J. Chromatography 403: 197-206, herein incorporated by reference. Proteins were dissolved in 20 mM sodium phosphate buffer containing 1.0 M sodium chloride at pH 7.5, to provide a concentration of either 1 mg protein/ml or 0.5 mg protein/ml. The UV absorbance of the proteins at this concentration were measured at a wavelength of 280 nm.

Each column was then sequentially washed with 10 column volumes of 0.1 M EDTA at pH 7.0 followed by 10 column volumes of deionized water. The adsorbent in each column was then charged with 4 column volumes of 50 mM copper sulfate. The excess copper ions were removed by washing with 10 column volumes of deionized water or more until no more copper ions were detected in the wash water. Each column was then equilibrated with 10 column volumes of 20 mM sodium phosphate containing 1.0 M NaCl at pH 7.5. The protein solution was then continuously loaded onto each copper-loaded column and 1 or 0.5 ml fractions of the elution were collected and analyzed by UV. When the LTV absorbance at 280 nm of the eluant was one-half that of the original protein solution, the protein capacity of each column was determined by the difference in protein retention between the copper-free and the copper-loaded columns. The results for the various protein and peptide solutions is summarized in Table 1, below.

Peptide solution Preparation

Lactate dehydrogenase (LDH) was isolated from chicken breast muscle and the peptides were cleaved according to methods as described below, and in Chaga et al.(1992), “Purification and Determination of the Binding Site of Lactate Dehydrogenase from Chicken Breast Muscle on lmmobilized Ferric Ions,” J. Chromatography 627: 163-172, herein incorporated by reference. The resulting peptide mixture had 9 peptides, one of which is 3400 Daltons and binds nickel under specified conditions.

The peptide mixture was prepared as follows. 25 g of frozen chicken breast muscle were cut and placed in a blender with 120 ml of cold 50 mM sodium phosphate containing 1.0 mM EDTA, 1 mM magnesium acetate, and 1.0 mM mercaptoethanol at pH 7.5. The mixture was blended for an additional 30 seconds.

The mixture was then divided into 4 centrifuge tubes and centrifuged for 30 minutes at 10,000 rpm. The supernatant solution was loaded onto a 450 ml SEPHADEX G-25 (medium) gel filtration column equilibrated with 20 mM sodium phosphate buffer containing 1.0 M NaCl and 60 mM imidazole at pH 7.0. The protein extract was collected, then loaded onto a 40 ml chelating SEPHAROSE FF column charged with nickel and equilibrated with 20 mM sodium phosphate buffer containing 1.0 M NaCl and 60 mM imidazole at pH 7.0. The purified LDH mixture prepared in this manner was eluted from the column with 20 mM sodium phosphate buffer containing 1.0 M NaCl and 0.3 imidazole at pH 7.0. The salt and imidazole concentrations were reduced in the LDH mixture by ultrafiltration.

Trifluroacetic acid (TFA) was added to the LDH mixture in a 250 ml round bottom flask, until 70 % TFA solution was obtained. The approximate volume of the solution was 24 ml. 200 mg of cyanogen bromide were added to this solution. The mixture was purged with argon, sealed, and placed in the dark for 24 hours. 1.25 ml of the cleaved mixture were piped into separate eppendorf tubes and maintained at −45 ° C. until utilized.

Determination of Peptide Capacity

The ability of the adsorbents of the present invention, described above, to retain the 3400 Dalton nickel binding peptide from the LDH peptide mixture was determined by standard chromatography methods. Each adsorbent was packed in a 3.5×0.5 cm I.D. column connected to a UV detector, a chart recorder, and a fraction collector. Each column was washed with 10 column volumes of deionized water at a flow rate of 1 cm/min (0.2 ml/min) then loaded with four column volumes of 50 mM nickel sulfate solution. The excess nickel was removed by washing each column with 10 column volumes of deionized water. Each column was then equilibrated with 10 column volumes of 20 mM sodium phosphate buffer containing 1.0 M NaCl and 60 mM imidazole at a pH of 7.0.

The TFA from the two eppendorf tubes of cyanogen bromide cleaved LDH was removed using a SPEEDVAC. The peptides from both tubes were combined and solubilized in 1 ml of deionized water. 025 ml of 20 mM sodium phosphate buffer containing 1.0 M NaCl and 0.3 M imidazole was added to the peptide mixture. The peptides were centrifuged and the supernatant was filtered (0.22 μm filter).

Portions of the peptide mixture were then loaded onto each column, equilibrated as described above, each of which was then washed with 18 column volumes of the equilibration buffer. 1 ml fractions of the eluant were collected. After this extensive washing, the UV absorbance of the eluant from each of the columns had returned to baseline as indicated by the chart recorder (i.e., no UV absorbing species were eluted from the column). The bound peptide was eluted from each column by increasing the concentration of imidazole in the equilibration buffer to 0.3 M. The ability of the adsorbent to bind the peptide was determined by reverse phase chromatography of the eluted fractions using a linear gradient of acetonitrile. The amount of bound peptide was quantified based on the integrated area ratios of peptide peaks in the initial mixture.

Copper Extraction from Human Plasma

The copper extracting properties from human plasma of the adsorbents of the present invention was measured by copper-free standard chromatography techniques. Each adsorbent was packed in a 3.5×0.5 cm I.D. column connected to a UV detector, a chart recorder, and a fraction collector. Each column was washed with 10 column volumes of deionized water a flow rate of 1 cm/min (0.2 ml/min) then equilibrated with 10 column volumes of 20 mM sodium phosphate buffer containing 0.25 M NaCl at a pH of 7.45.

11 ml of human plasma diluted ten times with the equilibration buffer containing 15 μmol of copper was loaded onto each column and 1 ml fractions were collected. After loading, each column was washed extensively with the equilibration buffer until baseline was obtained on the chart recorder (i.e., no additional copper was eluted). Elution of the column was performed with 0.2 M imidazole in the equilibration buffer. The collected fractions containing the breakthrough and elution peaks were evaluated for protein retention by size-exclusion chromatography using a BIO-SILECT SEC 400-5 column.

Each column was then extensively washed with approximately 10 column volumes of deionized water. Copper elution was performed by washing each column with 0.1 M EDTA at pH 7.0. The copper was collected and the concentration measured by a UV absorbance against copper standards.

### RESULTS

A summary of the protein and peptide retention capabilities of the NOVAROSE-IDA/PEG—CH₃ adsorbents of the present invention, prepared and evaluated as discussed above, are shown in Table 1, below. The results of optimizing separation conditions using a column packed with adsorbent #3 are shown in Tables 2 and 3, below.

TABLE 1
Summary of NOVAROSE-IDA/PEG-CH₃ Characterization.

| | | | LDH Pep | Cyto-c | RNase A | Albumin |
|---|---|---|---|---|---|---|
| | PEG | Cu²⁺ | 3.4 kD | 12.3 kD | 13.7 kD | 67 kD |
| Adsorbent | (μmol/g) | (μmol/ml) | (nmol/ml) | (nmol/ml) | (nmol/ml) | (nmol/ml) |
| #1 | 0 | 170 | 33 | 7805 | 1387 | 25 |
| #2 | 20 | 134 | 33 | 1463 | 438 | 7 |
| #3 | 50 | 113 | 3 | 24 | 11 | 0 |
| #4 | 100 | 87 | 0 | 0 | 0 | 0 |
| #6 | 200 | 73 | | 0 | 0 | 0 |

TABLE 2
Optimization of NOVAROSE-IDA/PEG-CH₃ Prepared with
50 μmol PEG/g Gel (#3) - Separation Conditions

| | Low | Normal | High |
|---|---|---|---|
| pH | | 7.0 | 7.5 |
| Imid | 0 | 60 mM | |
| Flow Rate | 0.33 cm/min | 1 cm/min | 2 cm/min |
| Salt | 0.25 mM | 1 M | |

TABLE 3
Optimization of NOVAROSE-IDA/PEG-CH₃ Prepared with
50 μmol PEG/g Gel (#3) - LDH Peptide Binding Capacities

| | Low | Normal | High |
|---|---|---|---|
| pH | | 3 nmol/ml | 0 nmol/ml |
| Imid | 16 nmol/ml | 3 nmol/ml | |
| Flow Rate | 2 nmol/ml | 3 nmol/ml | 1 nmol/ml |
| Salt | 2 nmol/ml | 3 nmol/ml | |

Cooper Capacities

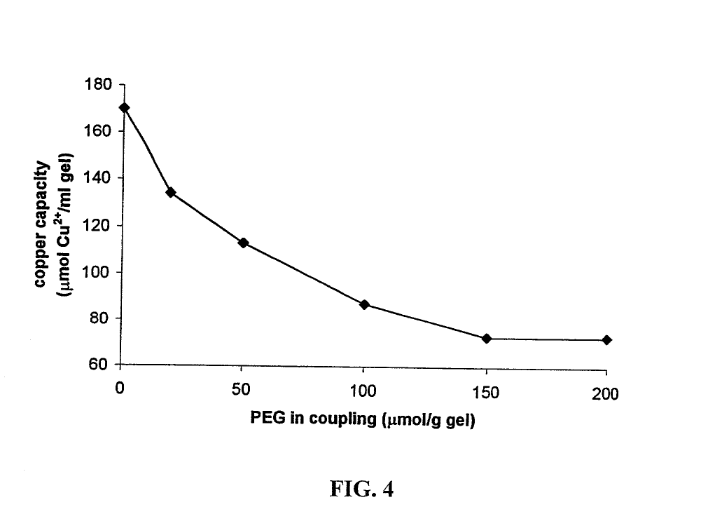

As expected, the copper capacities of the adsorbents of the present invention decreased as the concentration of the aminomonomethoxy-PEG on the adsorbent decreased (FIG. 4). The maximum copper capacity obtained for the control adsorbent (# 1) was 170 μmol Cu²⁺/ml gel. The adsorbent having the maximum quantity of PEG that could be coupled to the NOVAROSE (i.e., adsorbent #6) yielded a relatively high maximum copper capacity of 73 μmol Cu²⁺/ml gel.

Protein Capacities

Adsorbents that were exposed to more than 100 μmol aninomonomethoxy-PEG per gram of polymer matrix (i.e., gel) were unable to retain any proteins. Protein adsorption was only evident in adsorbents exposed to 50 μmol aminomonomethoxy-PEG or less per gram of gel.

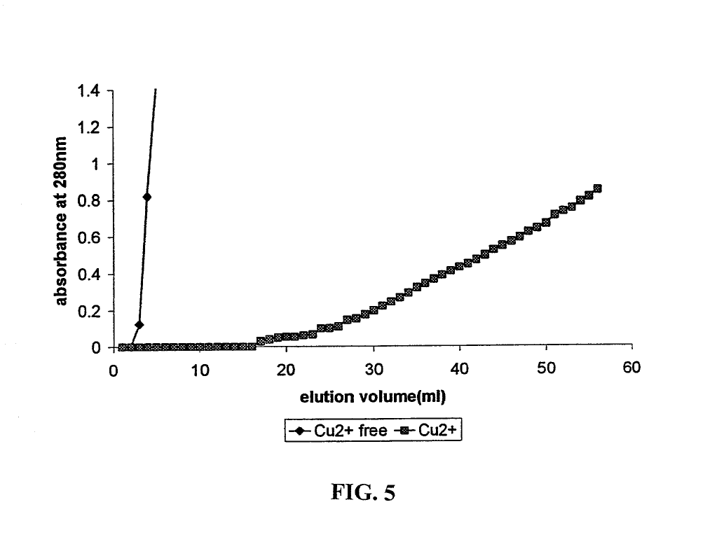

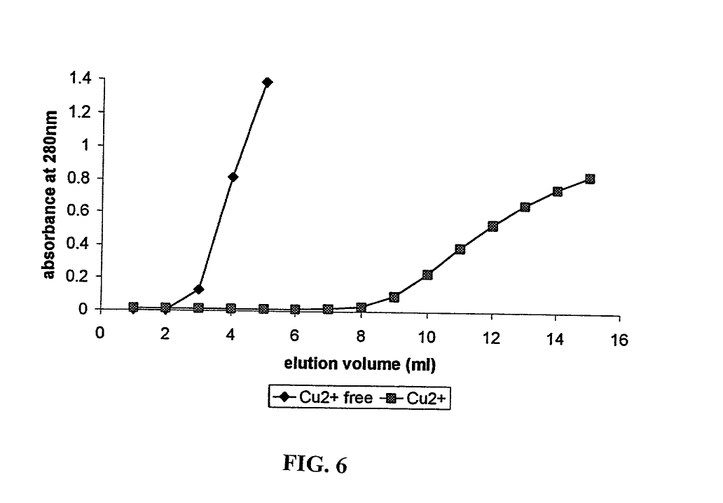

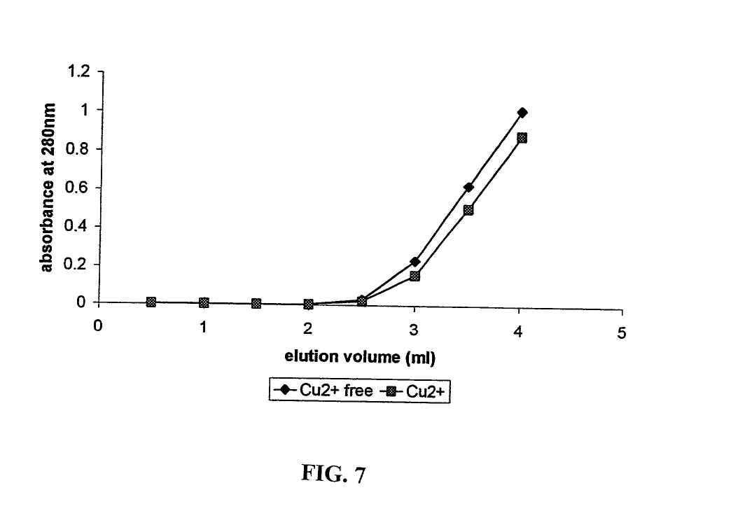

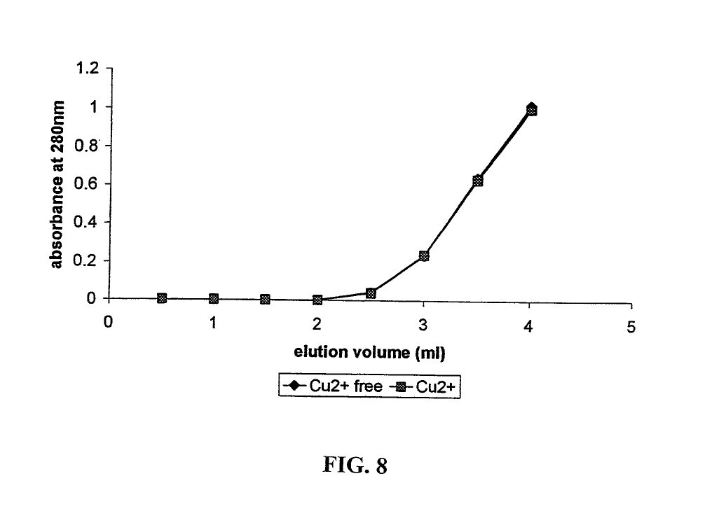

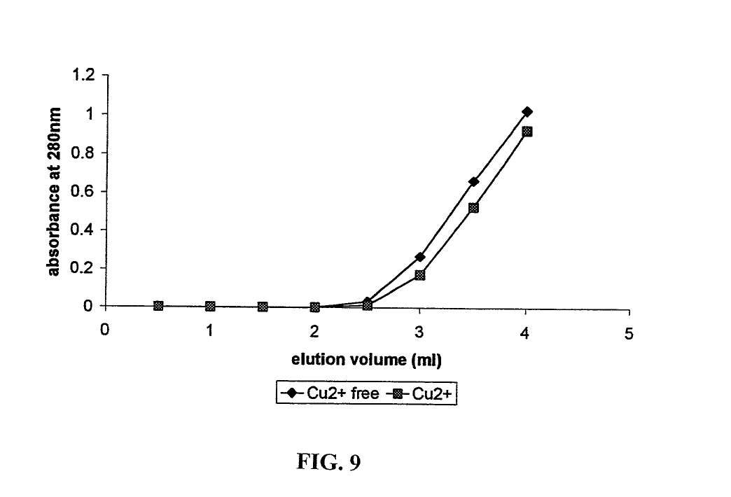

Cytochrome-c (12.3 kD) was the smallest protein used in frontal analysis of NOVAROSE-IDA/PEG—CH₃ adsorbents. The control adsorbent (# 1) was able to retain 7.8 μmol cytochrome-c per ml of adsorbent (FIG. 5). A decrease in copper capacity of 21% for the adsorbent exposed to 20 μmol PEG/g (# 2) resulted in a decrease in cytochrome-c binding of 81% (FIG. 6). A copper capacity decrease of 34% for the adsorbent exposed to 50 μmol PEG/g (# 3) lead to a cytochrome-c capacity decrease of 99% (FIG. 7). Adsorbents exposed to 100 μmol PEG/g (#4) and 200 μmol PEG/g (#6) were unable to adsorb cytochrome-c (FIGS. 8 and 9, respectively).

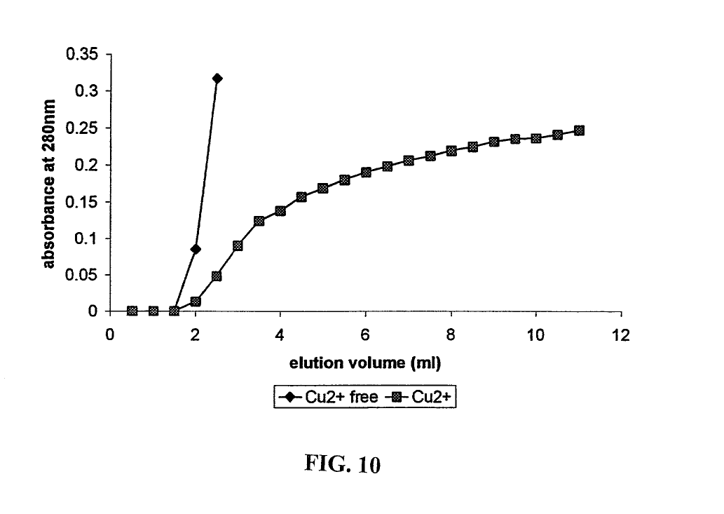

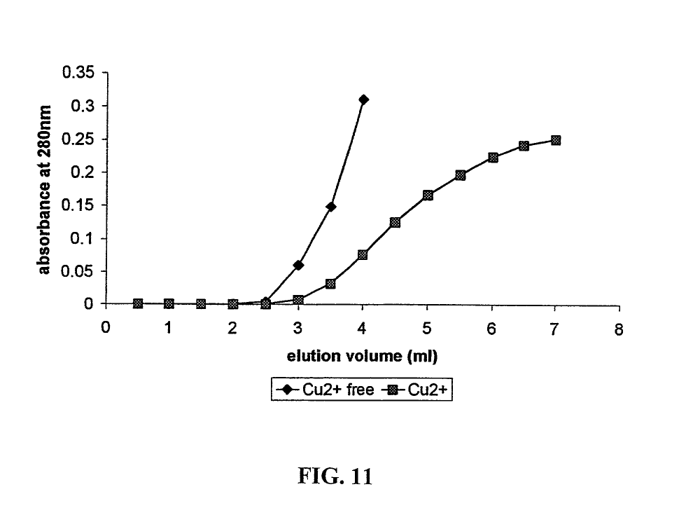

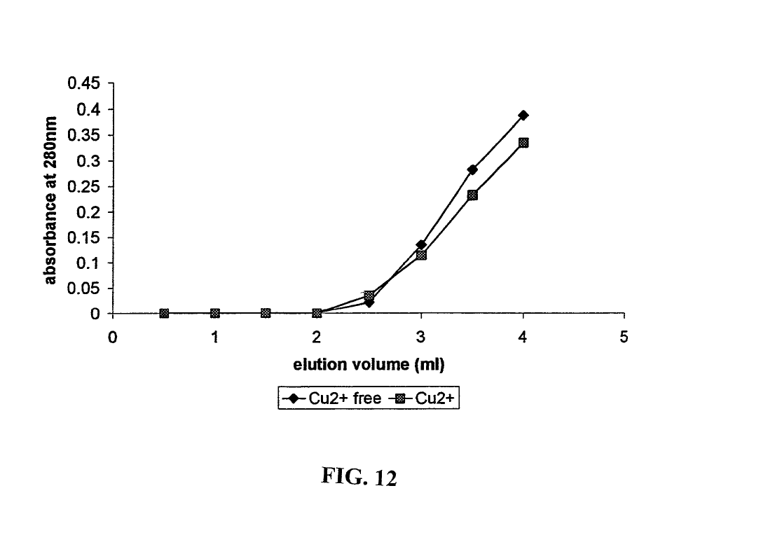

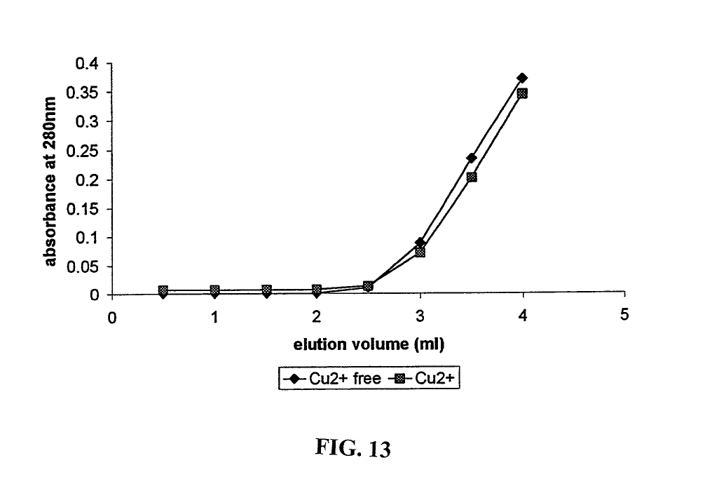

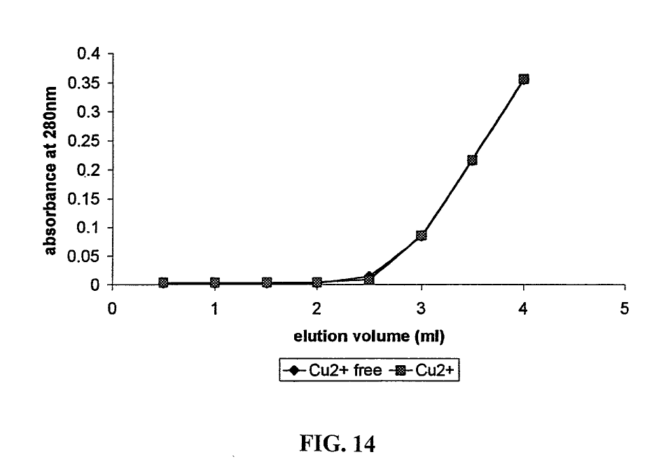

RNase A (13.7 kD) was also used to determine the ability of the adsorbents to bind protein. The control adsorbent (# 1) was able to bind 1.4 μmol RNase A per milliliter of adsorbent (FIG. 10). This protein retention capacity decreased 6.8% (FIG. 11) for the adsorbent exposed to 20 μmol PEG/g (# 2). The protein capacity decreased further by 99% from the control (FIG. 12) for the adsorbent exposed to 50 μmol PEG/g (# 3). Adsorbents exposed to 100 μmol PEG/ml (# 4) and 200 μmol PEG/g (# 6) were unable to adsorb RNase A (FIGS. 13 and 14, respectively).

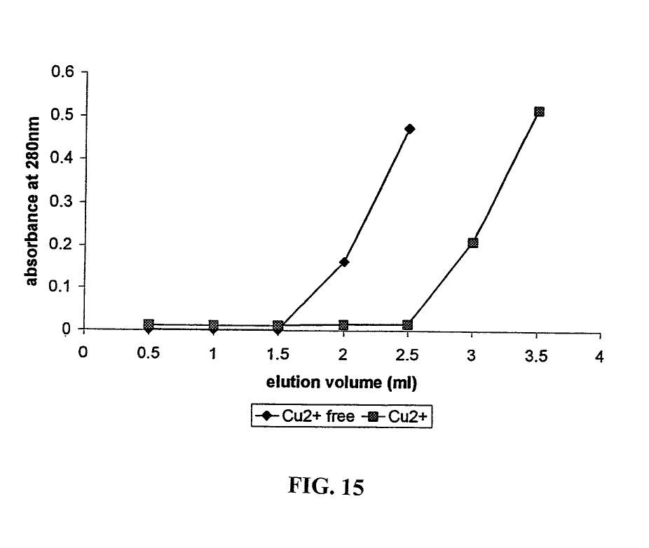

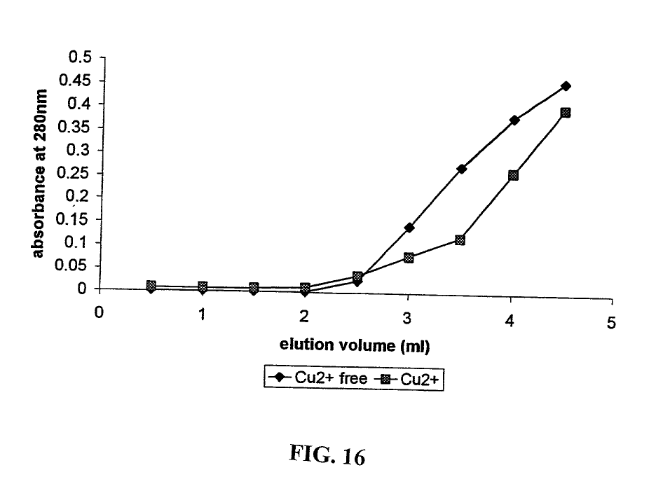

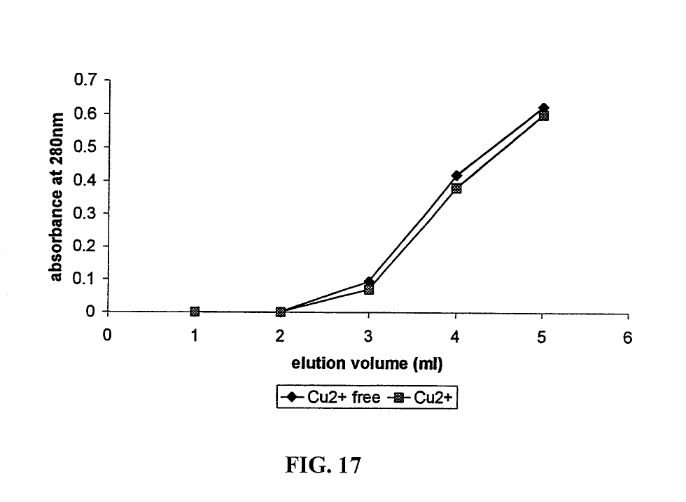

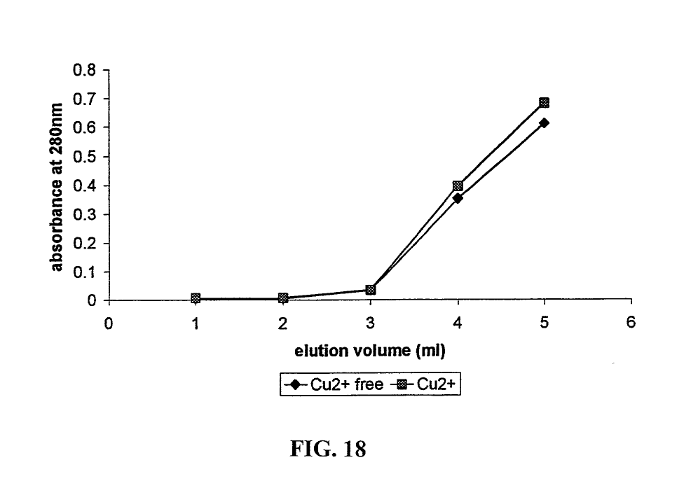

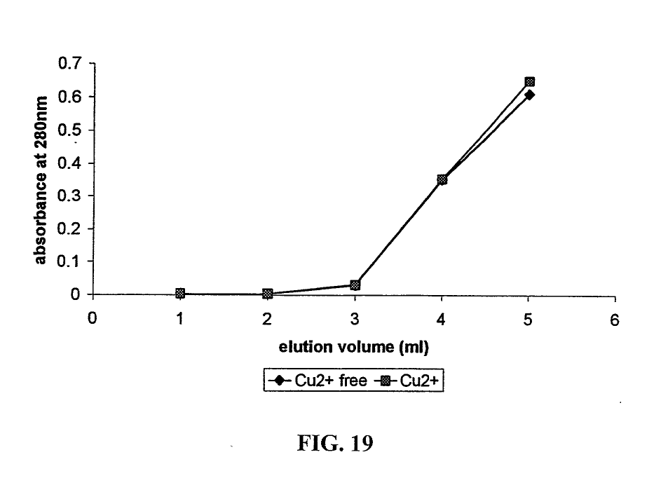

Albumin (67 kD) was the largest protein used for frontal analysis on these adsorbents. The control adsorbent (# 1) was able to retain 25 μmol of albumin per milliliter of gel (FIG. 15). The adsorbent exposed to 20 μmol PEG/g (# 2) was able to bind 7 nmol albumin per milliliter gel (FIG. 16). All other adsorbents were unable to retain Albumin (FIGS. 17, 18, and 19). It must be noted that adsorbent # 3 was able to slightly bind to albumin, but it was too small to be quantified (FIG. 17).

Peptide Capacities

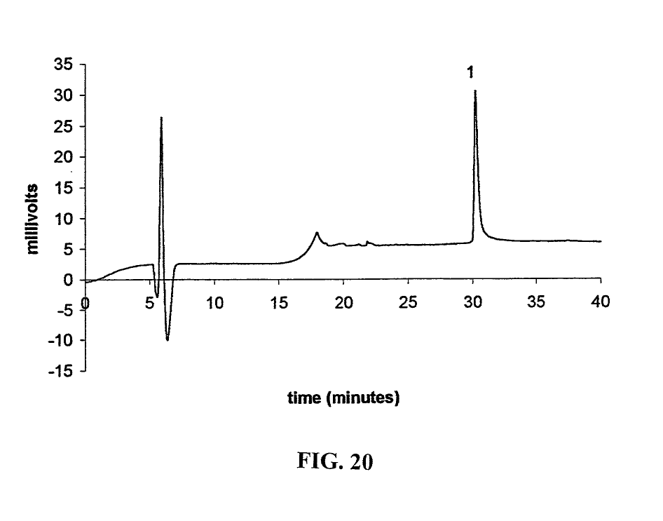

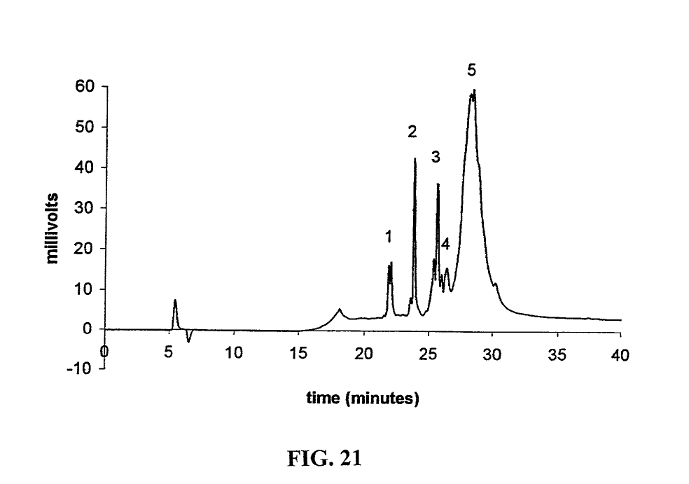

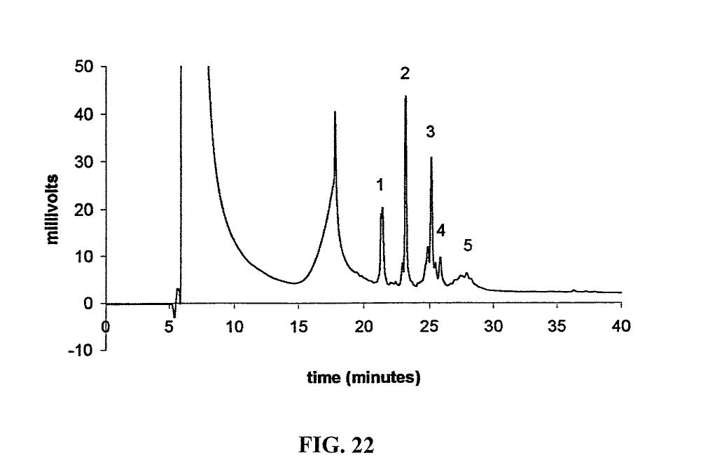

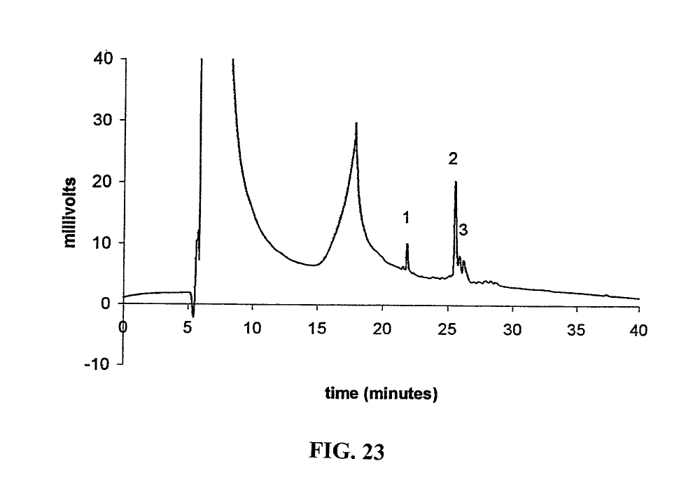

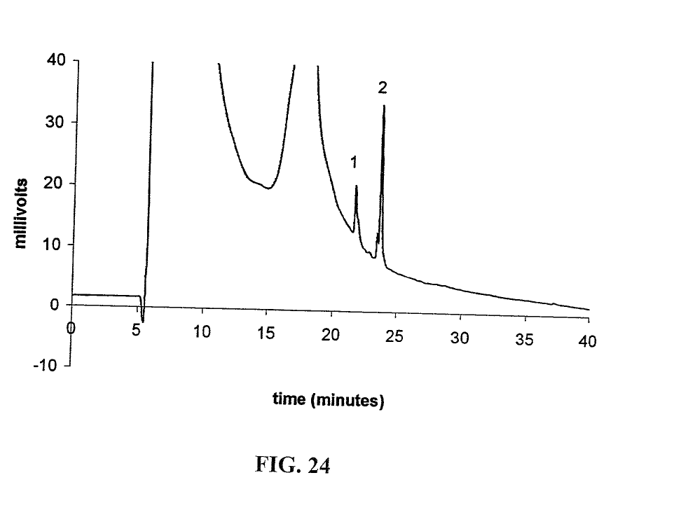

Lactate dehydrogenase (LDH) was isolated from chicken breast muscle using the procedures described above (FIG. 20). Cleavage of LDH by cyanogen bromide yielded an assortment of peptides (FIG. 21). Solubilization of the peptide mixture in 60 mM imidazole resulted in the precipitation of one peptide, peak 5 (FIG. 22). TNBS (i.e., trinitrobenzylsulfonic acid) measurement of the free amino groups of the peptide mixture indicated that each 1.25 ml eppendorf tube contained approximately 10 nmol of each peptide. The nickel binding target peptide was determined by the standard chromatography on Chelating Sepharose FF as performed previously in Chaga et al.(1992), “Purification and Determination of the Binding Site of Lactate Dehydrogenase from Chicken Breast Muscle on Immobilized Ferric Ions,” J. Chromatography 627: 163-172. The breakthrough peaks are indicated in FIG. 23 and the elution peaks are shown in FIG. 24. Both peaks (labeled, respectively as “1” and “2”) in FIG. 24 were isolated and amino acid analysis performed. Peak 2 was found to be the target 3400 D nickel binding peptide while peak 1 was unidentifiable.

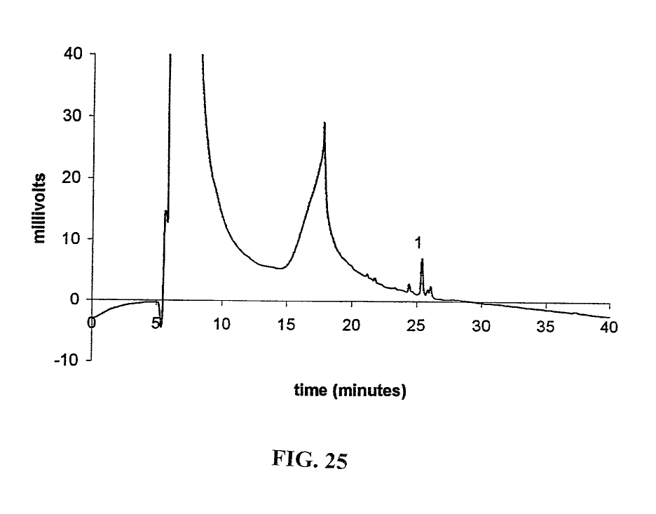

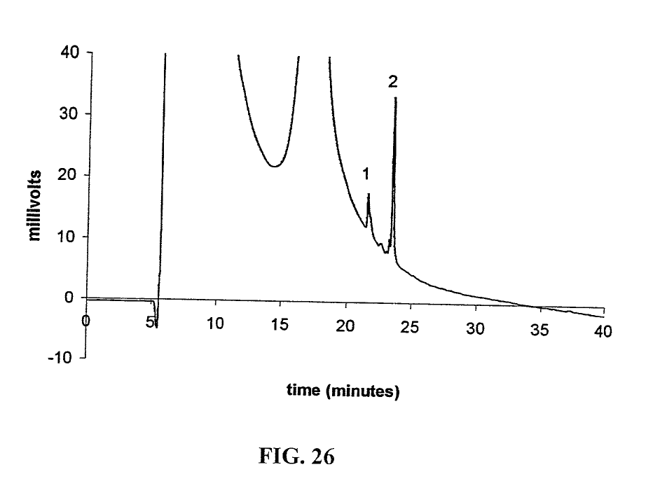

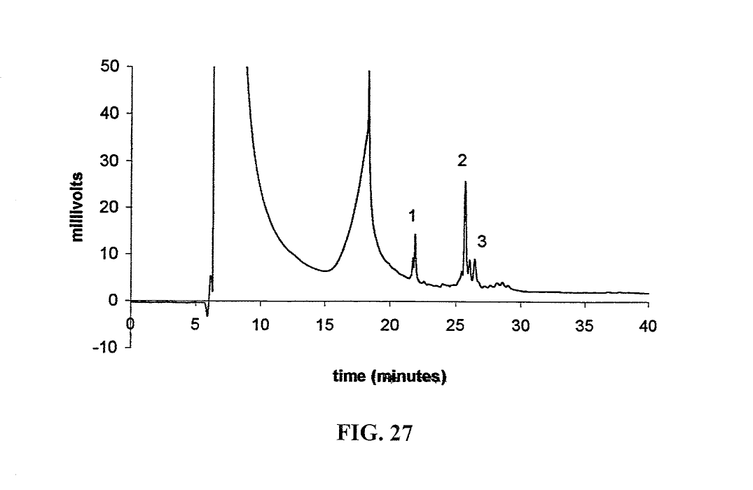

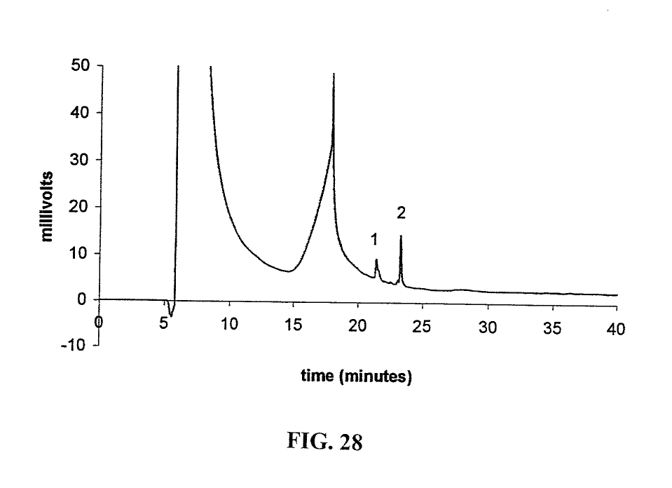

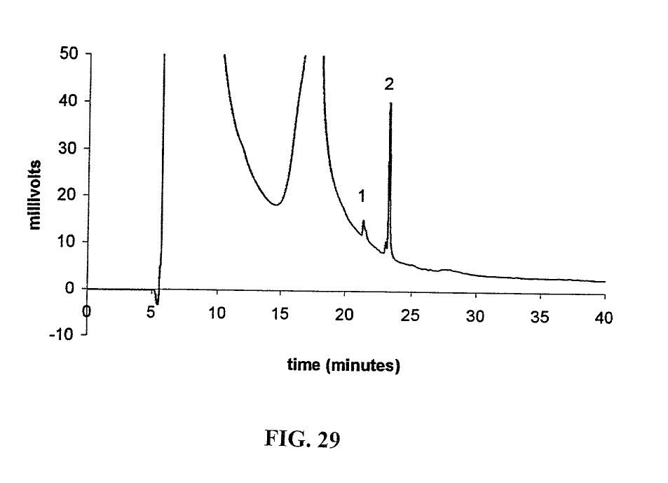

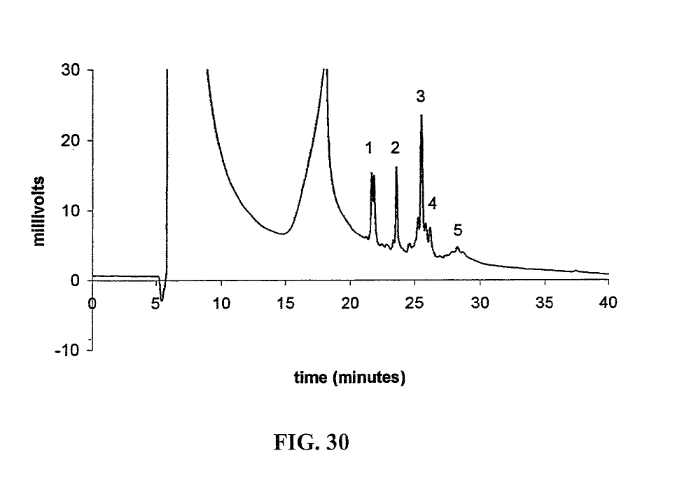

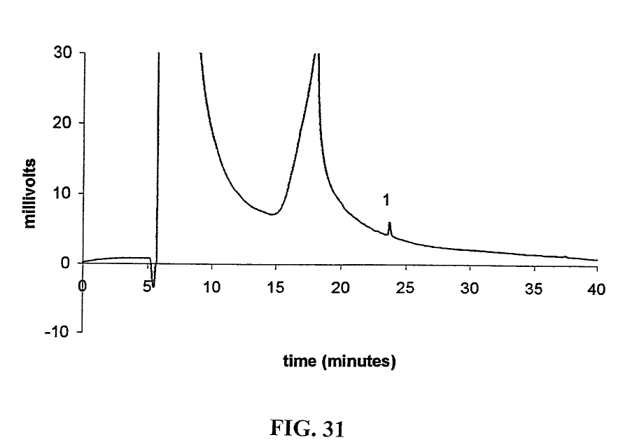

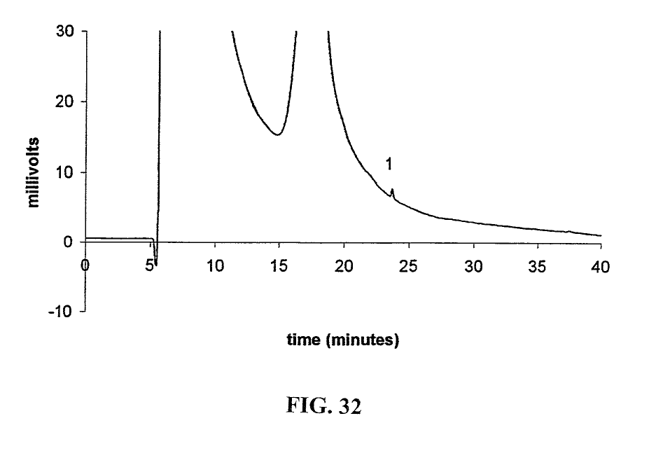

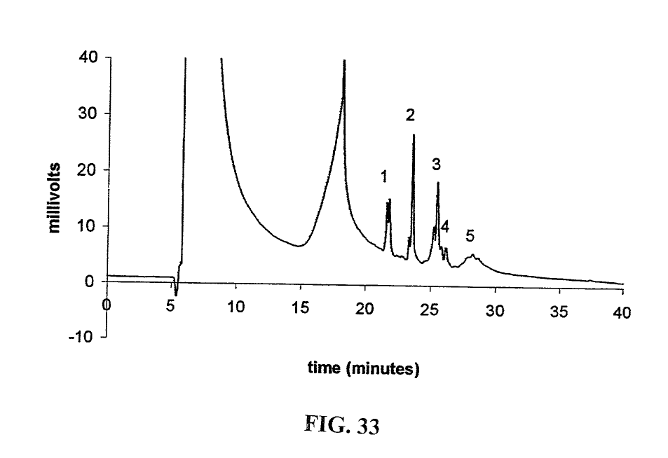

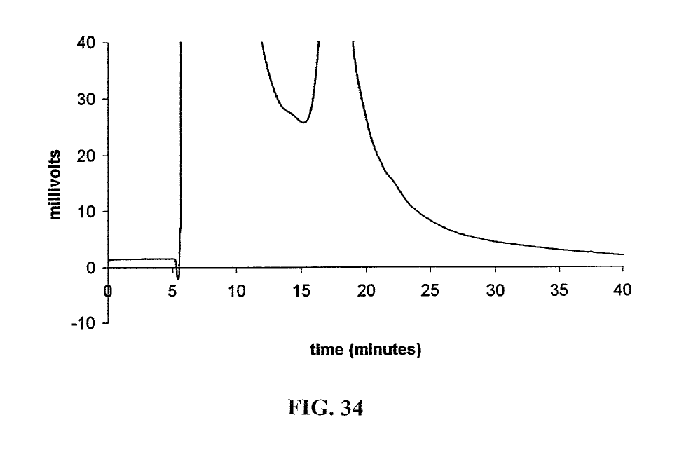

20 nmol of peptide solution were then loaded on each adsorbent, beginning with the control (# 1), to measure retention capabilities of the target peptide. 0.6 ml of the control adsorbent (# 1) was able to retain all 20 nmol of peptide. RPC of the breakthrough peak can be found in FIG. 25 and the elution peak containing the target peptide (peak “2”) can be found in FIG. 26. 0.6 ml of the adsorbent exposed to 20 μmol PEG/g gel (# 2) was able to retain all 20 nmol of the target peptide. The breakthrough is represented by FIG. 27. The elution peaks were divided into two tubes represented by FIGS. 28 and 29. The adsorbent exposed to 50 μmol PEG/g gel (# 3) was only able to retain 3 nmol of the 20 nmols of peptide loaded (FIGS. 30, 31, and 32). 0.98 ml of the adsorbent exposed to 100 μmol PEG/g gel (# 4) was unable to retain any peptide (FIGS. 33 and 34).

Optimization of Peptide Adsorption

Absorbent # 3 was able to bind 3 nmol of peptide per milliliter of gel, and was selected as the absorbent for a study to optimize peptide binding conditions. The various conditions studied are summarized in Table 2, above, and the resulting peptide binding capacities are summarized in Table 3, above. Only one of the standard chromatography conditions was altered at a time.

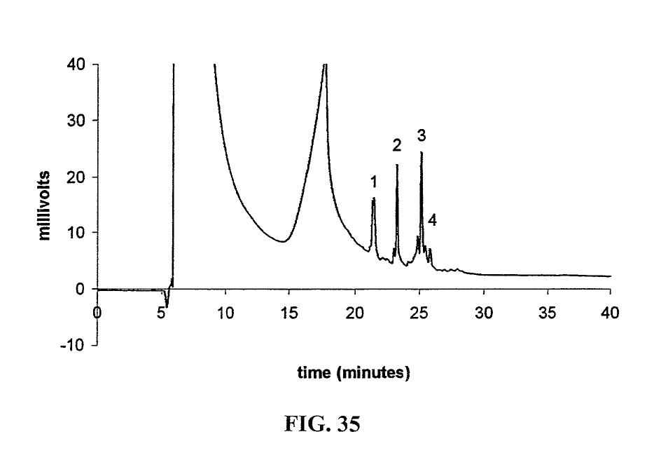

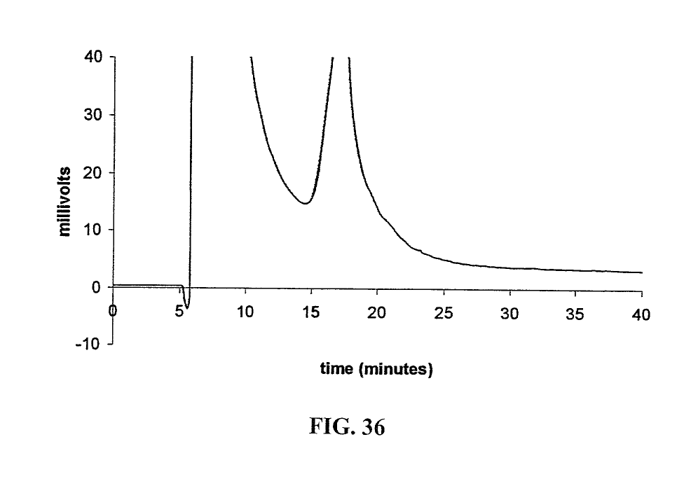

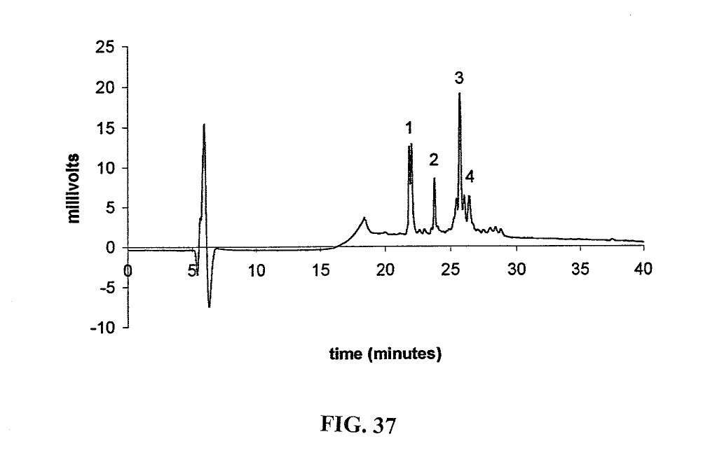

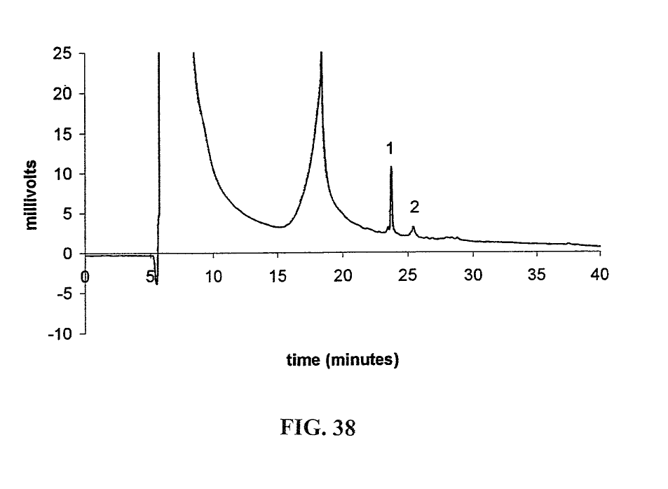

Increasing the pH of the equilibration buffer from 7.0 to 7.5 prevented the peptide from being retained in the column (FIGS. 35 and 36). Reducing the concentration of imidazole from 60 nM to 0 nM resulted in an increase in peptide binding to 16 nmol/ml (FIGS. 37 and 38). An additional peptide was also bound because the specificity towards the target protein decreased with the absence of imidazole.

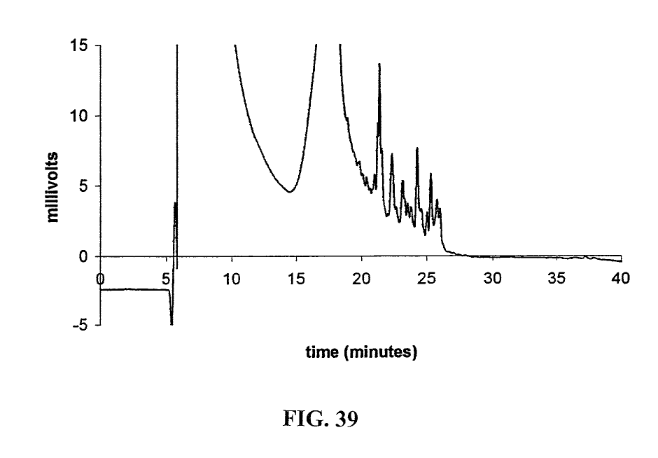

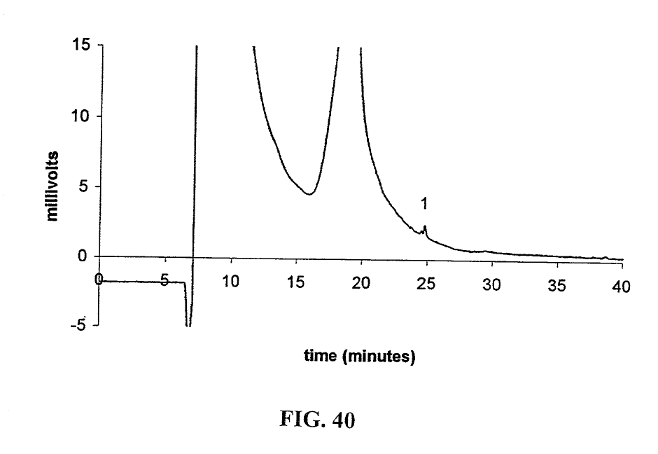

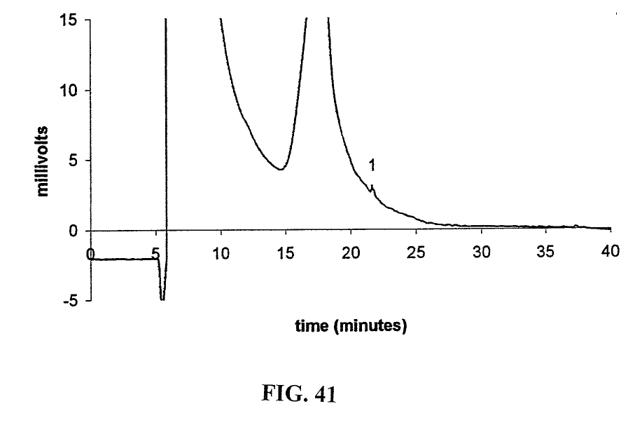

An investigation of diffusion limitations was carried by lowering the flow rate in the column from 1 cm/min to 0.33 cm/min. The low flow rate conditions resulted in the retention of only 2 nmol of peptide on the 0.98 ml column (FIG. 39, 40 and 41). FIG. 39 demonstrates that degradation of peptides seemed to also occur under these operational parameters.

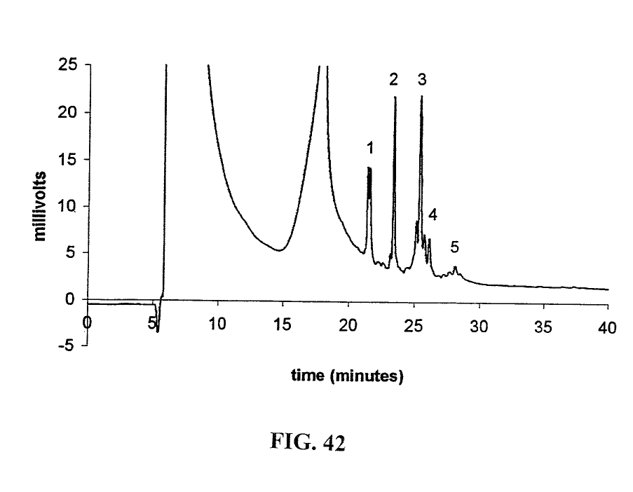

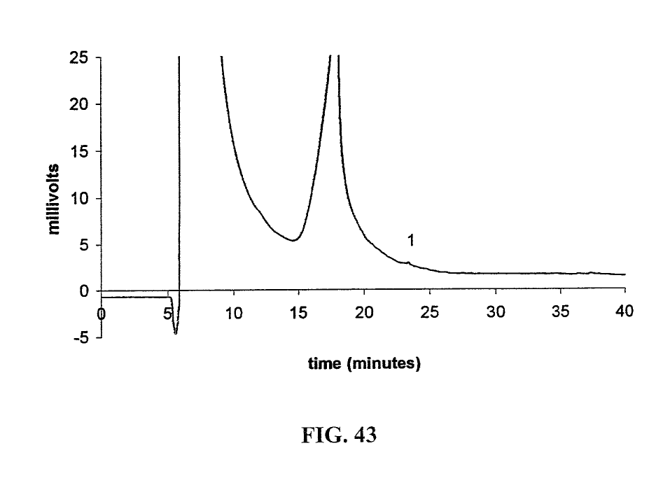

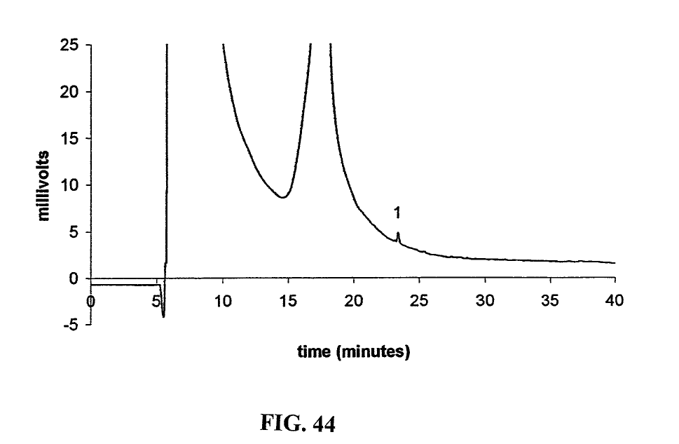

A flow rate of 2 cm/min was also investigated. The increase in flow rate caused a decrease in peptide adsorption to 1 nmol/ml (FIGS. 42, 43 and 44).

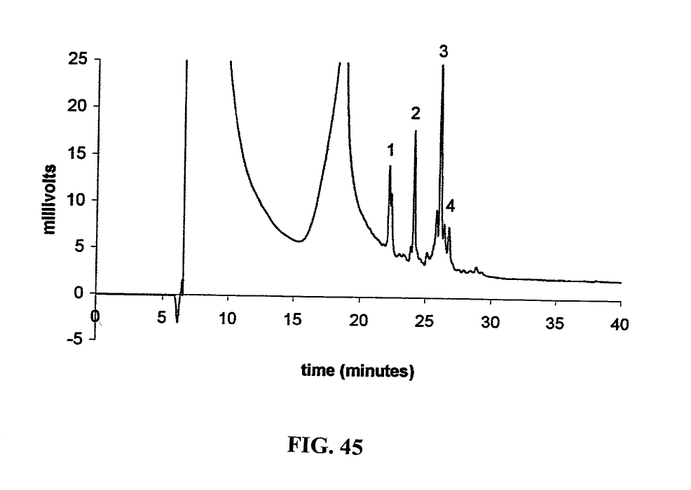

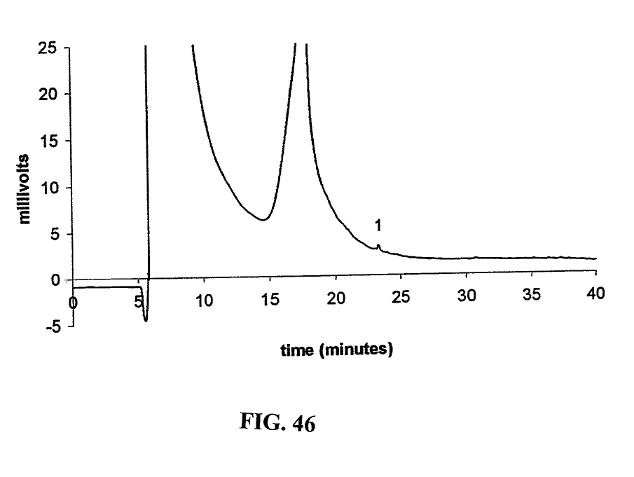

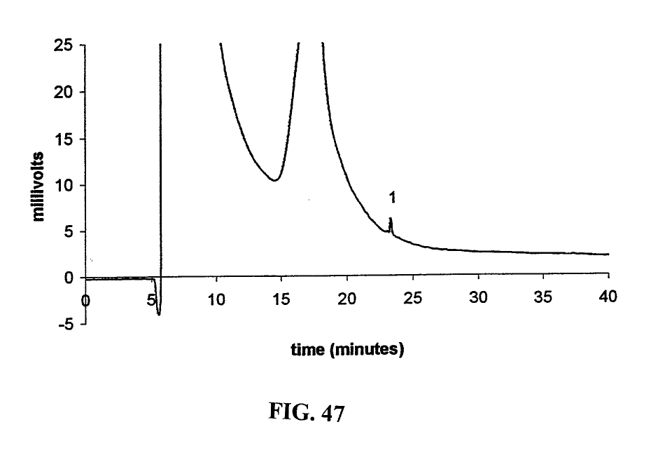

The salt concentration in the equilibrium buffer was also lowered from 1 M NaCl to 0.25 M NaCl. Only 2 nmol of peptide were able to bind to the 0.98 ml column under these conditions (FIG. 45, 46 and 47).

Copper Extraction from Human Plasma

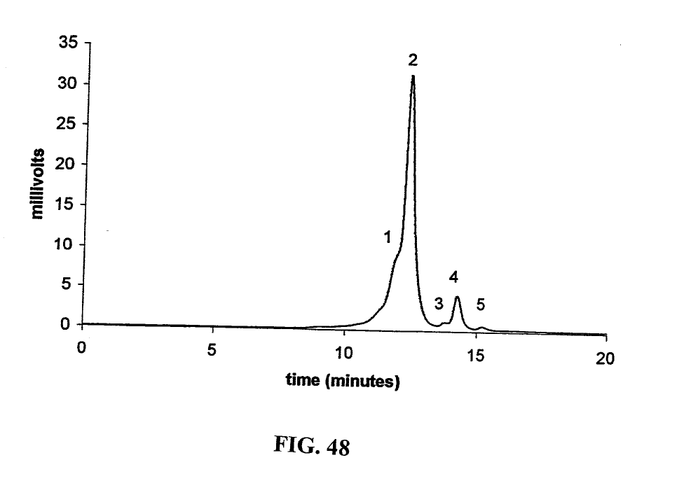

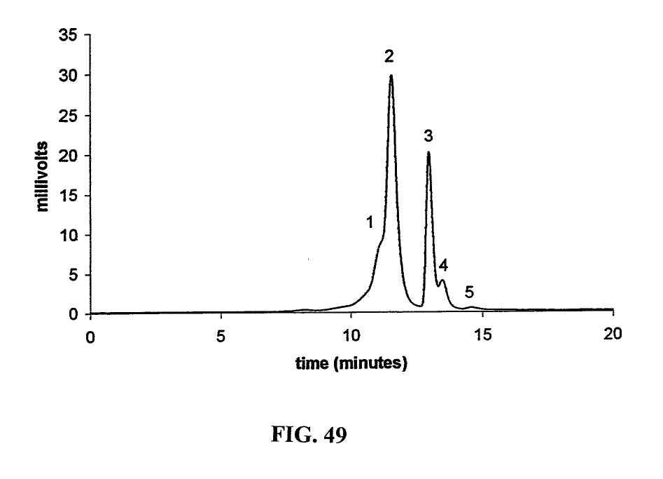

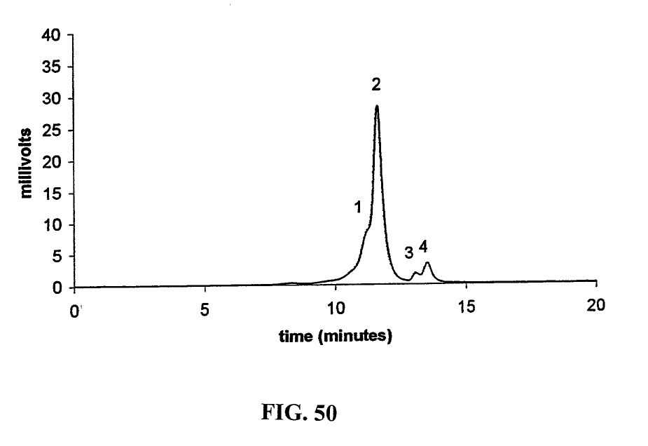

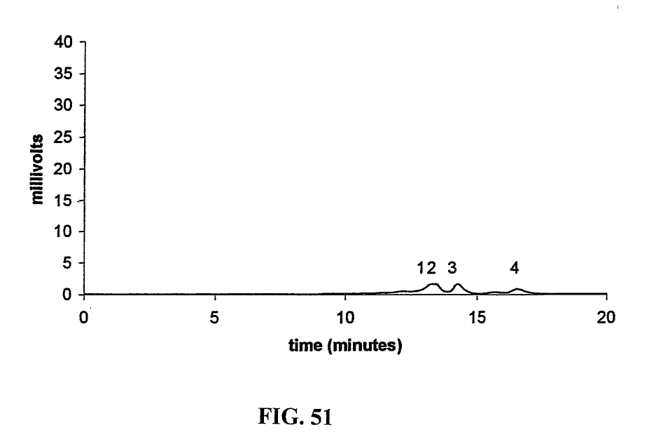

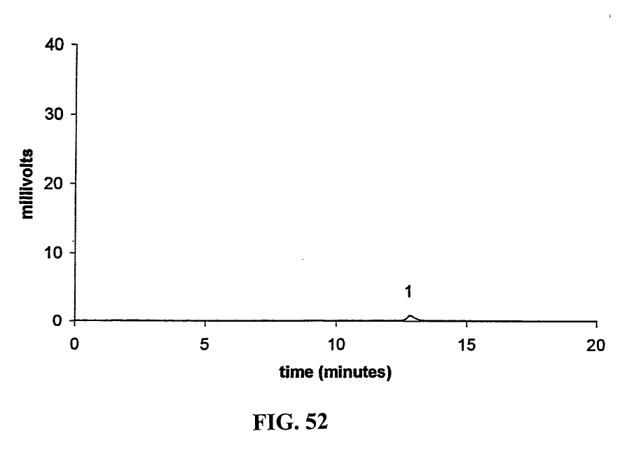

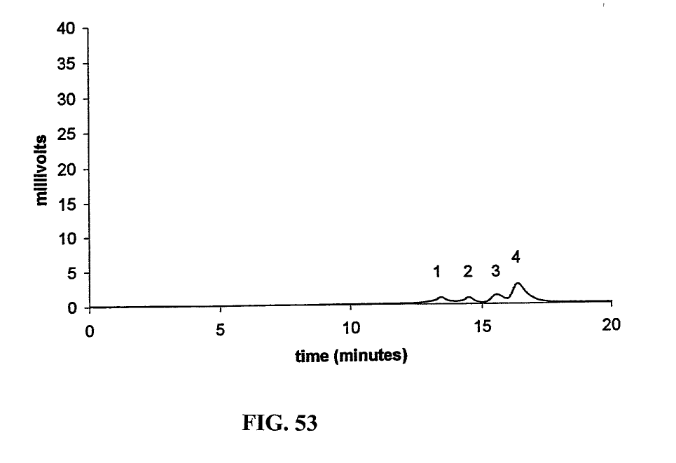

A sample of ten-fold diluted plasma was analyzed initially by size exclusion chromatography (SEC) (FIG. 48). If 15 μmol of copper is added to the diluted plasma, an additional peak in the SEC chromatogram appears, which corresponds to the copper ion or protein complexes resulting from the presence of copper (i.e., peak #3, FIG. 49). The adsorbent selected for this study was adsorbent # 6, prepared with 200 μmol PEG/g of gel, since adsorbent #6 exhibited copper binding, yet no protein or peptide binding. The SEC chromatogram of the breakthrough peak of human plasma with this adsorbent showed a decrease in peak 3 in the presence of copper (FIG. 50). The SEC chromatogram of the eluant showed four small peaks (FIG. 51). The SEC chromatogram of an equilibration buffer containing 0.2% Cu²⁺ (FIG. 52) showed that peak 1 of FIG. 51 is due to the equilibration buffer, and the SEC chromatogram of the elution buffer (FIG. 53) shows that peaks 2,3, and 4 are due to the elution buffer.

A comparison of the UV absorbance of the Cu²⁺ eluted from the column with an EDTA solution with Cu²⁺-EDTA standards showed that the adsorbent # 6 column having a volume of 0.59 ml was able to retain 87% of the copper loaded.

The aminomonomethoxy-PEG (5000 D) shielding ligand attached to the NOVAROSE 100/40 Act^{high} gel at a maximum density (i.e., adsorbent #6) resulted in a significantly high minimum copper capacity of 73 μmol Cu²⁺/ml gel. This copper capacity was much greater than the minimum copper capacity of 36 μmol Cu²⁺/ml gel obtained for similar diamino-PEG adsorbents in which the aminomonomethoxy-PEG shielding ligand described above was replaced with a diamino-PEG. Examples of adsorbents in which the shielding ligand is prepared from a diamino-PEG are described in detail below. Two factors probably account for the different copper capacities of the two types of adsorbents. The aminomonomethoxy-PEG, as described above, is coupling to the polymer matrix (i.e., gel) in an end-on configuration so that other active sites are not occupied by bridging amino groups. The aminomonomethoxy-PEG is a bulkier molecule than the diamino-PEG, and therefore fewer molecules are required to obtain maximum packing density. Accordingly, much smaller quantities of aminomonomethoxy-PEG were required on the gel surface to result in dramatic decreases in protein and peptide binding capabilities. This is probably a result of the relative steric bulk of the 5000 D PEG molecule, which apparently provides sufficient steric hindrance to inhibit access by proteins and peptides to available metal chelate sites.

The study of peptide binding to adsorbent # 3 in which various separation conditions were optimized, showed that the peptide binding conditions such a pH, flow rate, and salt concentration described in Chaga et al.(1992), “Purification and Determination of the Binding Site of Lactate Dehydrogenase from Chicken Breast Muscle on Immobilized Ferric Ions,” J. Chromatography 627: 163-172 were optimal for the adsorbents of the present invention. If imidazole was absent from the equilibration buffer, the peptide binding capacity of adsorbent #3 increased by 81%. This suggests that the peptides were able to diffuse through the PEG layer and interact with the immobilized metal in the absence of imidazole, but if imidazole was present, peptide retention was prevented. It therefore appears that imidazole serves as a competitive molecule for metal binding sites. Its presence increases selectivity for the high affinity nickel binding 3400 D peptide.

The adsorbents having the greatest packing density of aminomonomethoxy-PEG were unable to bind protein or peptide, yet, maintained a high capacity for metal ions. Suitable applications for these adsorbent may therefore be, for example, detoxification of protein solutions. Specifically, the experiments described above in which 15 μmols of copper was extracted from human plasma by adsorbent # 6 show that the adsorbents according to the present invention may be used to treat Wilson's disease. Wilson's disease is an inherited disorder characterized by the inability to metabolize copper. The typical elevated copper concentrations in plasma of people afflicted with the disorder are approximately 30 nmol of copper per milliliter of plasma, as described in Smith, J. et al. (1985), “Analysis and Evaluation of Zinc and Copper in Human Plasma and Serum,” J. of Amer. Col. Nutrition 4: 627-638, herein incorporated by reference. The present clinical treatments for this disease include administration of copper chelating agents such as penicillamine and trientine, which eliminate copper slowly and have adverse side effects, as described in Ogihara, H. et al (1995), “Buffer Exchange using Size Exclusion Chromatography, Countercurrent Dialysis, and Tangential Flow Filtration: Models, Development, and Industrial Application,” Biotech. Bioeng. 45: 149-157, herein incorporated by reference. In patients having extremely elevated copper concentrations or who are unable to take available therapeutic agents, extracorporeal treatment of their blood with a high density NOVAROSE-IDA/PEG—CH₃ adsorbent, as described above, may be invaluable. Thus, devices for blood dialysis and blood perfusion systems using the adsorbents of the present invention (e.g., the apparatus of FIG. 3) may be used to treat Wilson—s disease patients

Adsorbents Derived from Diamino-PEG

Application to Adsorbents with Metal Ion Affinity.

Materials

Iminodiacetic acid (IDA), NaBH₄, Glycine, Na₂CO₃, and NaOH were purchased from Sigma, St. Louis, Mo.

NH₂—PEG—NH₂ (0,0′-Bis(2-aminopropyl)polyethylene glycol) MW=1900 D was purchased from Fluka, Ronkonkoma, N.Y. (Cat. # 14529).

NOVAROSE ACT^{(High)} SE 100/40 was obtained from Inovata AB, Stockholm, Sweden.

Human plasma was obtained from the American Red Cross, Tucson, Ariz.

Methods

Coupling of various ratios of PEG/IDA:

20 g suction dried NOVAROSE ACTED) SE 100/40, 10 mL of 1.0 M Na₂CO₃ and 10 mL deionized water were mixed to form a slurry. The slurry was divided into four 50 ml tubes numbered 7-10.

2mL of 20% IDA (3 mmol of IDA) were added to tube # 7 containing 5 mL of the activated NOVAROSE gel, 25 mL of 1.0 M Na₂CO₃, 2.5 mL de-ionized water and 2 mL of 20% IDA in 1.0 mL of 10% NH₂—PEG—NH₂ in 1.0 M Na₂CO₃, at a pH≧12 were added to tubes 8, 9 and 10, and the tubes were shaken on a shaker.

After one hour, tube # 8 was centrifuged, the supernatant was removed, the gel was reconstituted in 25 mL of 1.0 M Na₂CO₃, 2.5 mL deionized water and 2 mL of 20% IDA in 1.0 M Na₂CO₃, at a pH≧12. The tube was again shaken.

After four hours, tube # 9 was centrifuged, the supernatant was removed, the gel was reconstituted in 25 mL of 1.0 M Na₂CO₃, centrifuged and the supernatant was removed again. To the gel were added 2.5 mL Na₂CO₃, 2.5 mL deionized water and 2 mL of 20% IDA in 1.0 M Na₂CO₃, at a pH≧12. The tube again shaken.

After 24 hours tube # 10 was centrifuged, the supernatant was removed, the gel was reconstituted in 25 mL of 1.0 M Na₂CO₃, centrifuged and the supernatant was removed again. To the gel were added 2.5 mL Na₂CO₃, 2.5 mL deionized water and 2 mL of 20% IDA in 1.0 M Na₂CO₃, at a pH≧12. The tube was again shaken.

After 48 hours, all of the tubes were centrifuged, the gels were washed with 25 mL of 1.0 M Na₂CO₃, and approximately 1 g of glycine in 5 mL of 0.5 M Na₂CO₃ was added (the pH was adjusted to ≧12). The tubes were left on the shaker for another 24 hours.

After 72 hours, all of the tubes were centrifuged, the gels were washed with water, 1 M NaOH, water, 0.1 M HCL and water.

Application to separation of human plasma proteins:

Immobilized Metal Ion Affinity Chromatography on Adsorbents with Controlled

Permeability:

The gels (i.e., adsorbents) prepared as described above, were packed into 5×1 cm I. D. columns, washed with 5 column volumes of 20 mM CuSO₄ solution in deionized water and the excess copper ions were removed by washing each column with 5 more column volumes of de-ionized water. The columns were then equilibrated with 5 column volumes of 20 mM sodium phosphate containing 0.25 M NaCl, at pH 7.45.

22 mL of human plasma diluted five-fold with the equilibration buffer were loaded onto the columns and the non-adsorbed material was washed out with the equilibration buffer until the UV absorbance (at 280 nm) of the eluant showed that no additional non-adsorbed material was present.

The adsorbed material was then eluted with a mixture of 20 mM imidazole in the equilibration buffer.

Table 4 summarizes the properties of these adsorbents, as shown below.

TABLE 4

| | Cu(II) Capacity | Eluted material, | Eluted material, % |
|---|---|---|---|
| Gel | μmol/mL gel | AU | of loaded |
| #7 | 182 | 13.7 | 7.2 |
| #8 | 91 | 6.1 | 3.2 |
| #9 | 64 | 4.2 | 2.1 |
| #10 | 30 | 3.2 | 1.6 |

Analysis of the Eluted Components

The composition of the eluant from the four columns described above was analyzed by analytical SEC on a SUPEROSE 12 HR 10/30 column equilibrated with 20 mM Tris/HCl and 0.25 M NaCl at a pH 7.5. 200 μl of the eluant from the adsorption columns described above was loaded onto the analytical SEC column, and eluted at 0.4 ml/min flow rate.

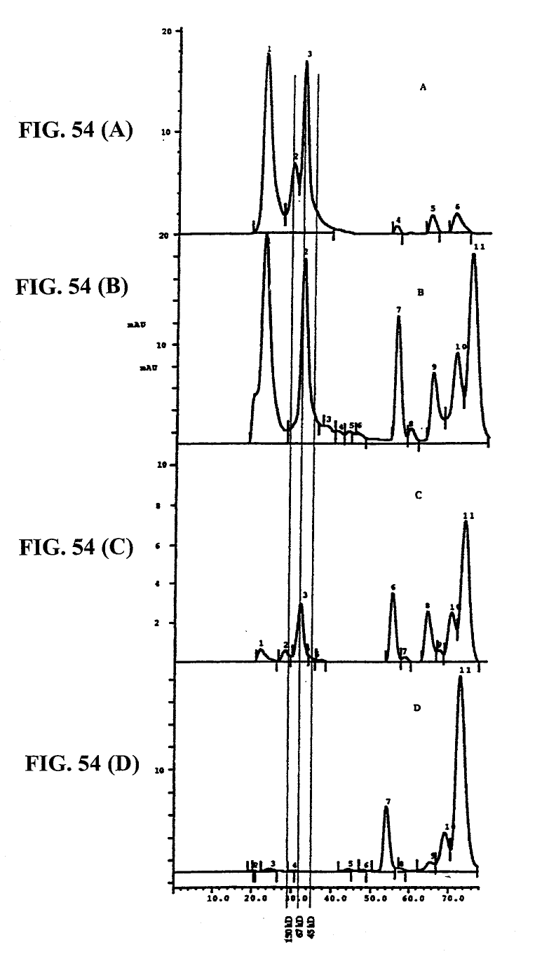

The elution profiles (FIGS. 54A-D) show that gels 8, 9 and 10 adsorbed successively lower molecular weight (M_{W}) compounds present in human plasma, showing that controlled adsorption has occurred. For example, while the control adsorbent #7 contained only 14.6% of Low Molecular Weight (LMW) compounds (M_{W} under 45 kD), the materials eluted from gels 8, 9 and 10 contained 54, 81 and 99% of LMW compounds, respectively.

The priority document of the present application, U.S. Provisional Application No. 60/289,576, filed May 7, 2001, is incorporated herein by reference.

What is claimed as new and is intended to be secured by letters patent is:

1. A polymer product comprising a matrix polymer, an affinity ligand, and a shielding ligand, wherein the affinity ligand forms complexes with small molecules and the matrix polymer, affinity ligand, and shielding ligand are covalently bonded together.

2. The polymer product of claim 1, wherein the affinity ligand and shielding ligand are both independently covalently bonded to the matrix polymer.

3. The polymer product of claim 1, wherein the affinity ligand is covalently bonded to the matrix polymer, and the shielding ligand is covalently bonded to the affinity ligand.

4. The polymer product of claim 1, wherein the matrix polymer is crosslinked.

5. The polymer product of claim 1, wherein the matrix polymer has the form of a gel.

6. The polymer product of claim 1, wherein the matrix polymer has the form of a membrane.

7. The polymer product of claim 1, wherein the matrix polymer has the form of a liposome.

8. The polymer product of claim 1, wherein the matrix polymer is selected from the group consisting of an insoluble polysaccharide, cellulose, cross-linked dextran, cross-linked agar, cross-linked agarose, and oxirane or halohydrin derivatives thereof.

9. The polymer product of claim 1, wherein the matrix polymer is an oxirane or halohydrin derivative of cross-linked agarose.

10. The polymer product of claim 1, wherein the affinity ligand is selected from the group consisting of a polyamine, a hydroxy aromatic, a salicylidene derivative derived from a salicyl aldehyde, a sulfide a sulfone, a metal complex of a polyamine, a metal complex of a hydroxy aromatic, a metal complex of a salicylidene derivative derived from a salicyl aldehyde, a metal complex of a sulfide, a metal complex of a sulfone, and mixtures thereof.

11. The polymer product of claim 1, wherein the affinity ligand is a polyamine.

12. The polymer product of claim 1, wherein the affinity ligand is prepared by covalently bonding iminodiacetic acid to the matrix polymer.

13. The polymer product of claim 1, wherein the affinity ligand is a metal ion chelate.

14. The polymer product of claim 13, wherein the metal is selected from the group consisting of nickel and copper.

15. The polymer product of claim 1, wherein the shielding ligand is a polyalkylene ether chain.

16. The polymer product of claim 1, wherein the shielding ligand is selected from the group consisting of polyethylene glycols, polypropylene glycols, polybutylene glycols, polytetramethylene glycols, and copolymers thereof.

17. The polymer product of claim 1, wherein the shielding ligand is end-capped with a neutral group.

18. The polymer product of claim 1, wherein the neutral group is s elected from the group consisting of a methyl group, and ethyl group, a propyl group, a butyl group, and a trialkylsilyl group.

19. A chromatography column comprising a cylinder packed with the polymer product of claim 1.

20. A method of separating a mixture of large and small molecular size solutes comprising contacting the mixture with the polymer product of claim 1, wherein at least a portion of the small molecular size solutes are selectively adsorbed by the affinity ligand.

21. The method of claim 20, wherein the mixture is a mixture of biological molecules

22. The method of claim 20, wherein the mixture comprises solutes selected from the group consisting of metal ions, metal ion complexes, immunoglobulins, microglobulins, antibodies, degradation products of antibodies, nucleic acids, antibiotics, toxins, drugs, hormones, and biotoxins.

23. The method of claim 20, wherein the mixture comprises blood plasma.

24. A method of extracorporeal perfusion of blood, comprising contacting the blood of a patient with the polymer product of claim 1, then returning said treated blood back to the patient.

25. A method of removing copper ions from a patient with Wilson's disease, comprising contacting the plasma of a patient having Wilson's disease with the polymer product of claim 12, wherein the shielding ligand is a monomethyl-PEG ether.

26. An apparatus for separating a mixture comprising small and large molecular size solutes, comprising:
a column packed with the polymer product of claim 1
means for introducing the mixture into the column, whereby at least a portion of the small molecular size solutes are selectively adsorbed by the polymer product
means for washing the unadsorbed solutes off of the column, and
means for desorbing the selectively adsorbed solutes off of the column.

27. A protein or peptide purified by contacting a mixture comprising the protein and at least one other protein with the polymer product of claim 1.

28. A method of preparing the polymer product of claim 1, comprising:
reacting a matrix polymer with an affinity ligand, thereby covalently bonding the affinity ligand to the matrix polymer; and
reacting a shielding ligand with the matrix polymer, thereby covalently bonding the shielding ligand with the matrix polymer.

29. A method of preparing the polymer product of claim 1, comprising:
reacting a matrix polymer with an affinity ligand, thereby covalently bonding the affinity ligand to the matrix polymer; and
subsequently reacting a shielding ligand with the affinity ligand bonded to the matrix polymer, thereby bonding the shielding ligand to the affinity ligand.

30. The method of claim 28, further comprising chelating the affinity ligand with a metal.

31. The method of claim 29, further comprising chelating the affinity ligand with a metal.
